(19) United States
(12) Patent Application Publication
(10) Pub. No.: US 2024/0301088 A1
(43) Pub. Date: Sep. 12, 2024

(54) CHIMERIC ANTIGEN RECEPTOR

(71) Applicant: AUTOLUS LIMITED, London (GB)

(72) Inventors: Martin Pulé, London (GB); John Anderson, London (GB); Simon Thomas, London (GB)

(21) Appl. No.: 18/515,458

(22) Filed: Nov. 21, 2023

Related U.S. Application Data
(63) Continuation of application No. 17/196,670, filed on Mar. 9, 2021, now Pat. No. 11,879,016.
Application No. 17/196,670 is a continuation of application No. 15/123,331, filed on Sep. 2, 2016, now Pat. No. 10,975,162.

(30) Foreign Application Priority Data
Mar. 6, 2014 (GB) 1403972.1

(51) Int. Cl. C07K 16/30; A61K 35/17; A61K 38/17; A61K 39/00; A61K 39/395; C07K 14/705; C07K 14/725; C12N 15/85

(52) U.S. Cl. CPC C07K 16/3084; A61K 35/17; A61K 38/1774; A61K 38/179; A61K 39/39558; C07K 14/7051; C07K 14/70517; C07K 14/70521; C07K 14/70578; C12N 15/85; A61K 2039/505; C07K 2317/21; C07K 2317/24; C07K 2317/52; C07K 2317/53; C07K 2317/565; C07K 2317/622; C07K 2317/73; C07K 2319/00; C07K 2319/03; C07K 2319/20; C07K 2319/30; C12N 2510/00

(57) ABSTRACT

Provision of a chimeric antigen receptor (CAR) comprising a disialoganglioside (GD2)-binding domain which comprises⋅a) a heavy chain variable region (VH) having complementarity determining regions (CDRs) with the following sequences:⋅b) a light chain variable region (VL) having CDRs with the following sequences: T cells expressing such a CAR are useful in the treatment of some cancers.

### CROSS-REFERENCE TO RELATED APPLICATIONS

This is a Continuation of U.S. application Ser. No. 17/196,670 filed Mar. 9, 2021, which is a Continuation of U.S. application Ser. No. 15/123,331 filed Sep. 2, 2016, which is a U.S. National Phase of International Application No. PCT/GB2015/50649, filed Mar. 6, 2015, which claims priority to Great Britain Application No. 1403972.1, filed Mar. 6, 2014.

### INCORPORATION BY REFERENCE OF THE SEQUENCE LISTING

This application contains, as a separate part of disclosure, a Sequence Listing in computer-readable form (Filename: 49341B_SeqListing.xml; 63,082 bytes—XML file dated Nov. 1, 2023) which is incorporated by reference herein in its entirety.

### FIELD OF THE INVENTION

The present invention relates to chimeric antigen receptor (CAR) which binds the cancer antigen disialoganglioside (GD2). T cells expressing such a CAR are useful in the treatment of cancerous diseases such as neuroblastoma.

### BACKGROUND TO THE INVENTION

Disialoganglioside (GD2, pubchem: 6450346) is a sialic acid-containing glycosphingolipid expressed primarily on the cell surface. The function of this carbohydrate antigen is not completely understood; however, it is thought to play an important role in the attachment of tumour cells to extracellular matrix proteins. GD2 is densely, homogenously and almost universally expressed on neuroblastoma. In normal tissues, GD2 expression is largely limited to skin melanocytes, and peripheral pain fibre myelin sheaths. Within the CNS, GD2 appears to be an embryonic antigen but is found dimly expressed in scattered oligodendrocytes and within the posterior pituitary. This makes GD2 well suited for targeted antitumour therapy.

Anti-GD2 antibodies have been extensively tested as therapy in neuroblastoma. Two clones and their derivatives are in current clinical use: clone 3F814 and 3F8. Another clone 14.187 has been tested as a mouse IgG3, after isotype switching to IgG2a (14g2a) and finally after chimerization with human IgG1 to form ch14.18. This latter antibody has resulted in clear efficacy in a randomized study: the US Children's Oncology Group reported a randomised phase III study of ch14:18 in children with high-risk neuroblastoma who had achieved radiological remission after initial treatment. In these patients, there was a 20% improvement in EFS in the ch14:18 arm with a mean follow-up of 2.1 years. Importantly, neurotoxicity most commonly as a chronic pain inducing neuropathy and less commonly an ophthalmoplegia is the main dose-limiting toxicity with these agents.

These therapeutic mAbs continue to be refined: an IL-2 immunocytokine derived from ch14.18 has been described. This is quite a toxic agent with some effect on minimal residual disease, but none against bulky disease. Ch14.18 has been fully humanized and its Fc mutated to inhibit complement activation. This humanized version of Ch14.18 is in clinical study but only very limited data are available. Humanization of the 3F8 antibody has also been described. While clinical data from GD2 serotherapy is encouraging, sustained complete remissions are still limited and there is no evidence for a clinically useful role for antibodies except in the minimal disease setting.

There is thus a need for improved therapeutic approaches to treat neuroblastoma and other GD2-expressing cancers.

### Chimeric Antigen Receptors (CARs)

Chimeric antigen receptors are proteins which, in their usual format, graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals (see FIG. 1a).

The most common form of these molecules are fusions of single-chain variable fragments (scFv) derived from monoclonal antibodies which recognise a target antigen, fused via a spacer and a transmembrane domain to a signaling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

Chimeric antigen receptors against GD2 have been described in which the antigen-binding domain is based on the scFv 14g2a (WO 2013/040371 and Yvon et al (2009, Clin Cancer Res 15:5852-5860)).

Human T cells expressing a 14g2a-CD28-OX40-ζ CAR was shown to have some anti-tumour activity but to be unable to completely eradicate the disease (Yvon et al (2009) as above).

The present inventors sought to make an alternative GD2-targeting CAR with improved properties.

### DESCRIPTION OF THE FIGURES

FIG. 1—Chimeric Antigen Receptor (CAR) design.

(a) generalized architecture of a CAR: A binding domain recognizes antigen; the spacer elevates the binding domain from the cell surface; the trans-membrane domain anchors the protein to the membrane and the endodomain transmits signals.

(b) to (d): Different generations and permutations of CAR endodomains: (b) initial designs transmitted ITAM signals alone through FcεR1-γ or CD3□ endodomain, while later designs transmitted additional (c) one or (d) two co-stimulatory signals in cis.

FIG. 2—Variants of anti-GD2 CARs constructed (a) anti-GD2 CAR using mouse KM666 antibody as scFv with human IgG1 spacer and CD28-OX40-Zeta endodomain; (b) anti-GD2 CAR using Nakamura humanized antibody huKM666 in the same format as (a); (c) same format as (b) except Fc domain is modified to remove Fc Receptor recognition motifs; (d) same format as (c) except spacer is IgG1 hinge-CD8 stalk; (e) same as (c) except spacer is CD8 stalk only; (f) same as (c) except spacer is IgG1 hinge only.

FIG. 3—Comparison of muKM666 and huKM666 based CARs. (a) Expression on peripheral blood T-cells from 3 normal donors; (b) mean-fluorescent intensity of these facs plots shown as a histogram; (c) Chromium release assay using non-transduced, muKM666 and huKM666 transduced T-cells as effectors against A204 (GD2 negative), and LAN-1 (GD2 positive) targets; (d) IL-2 production from the same challenge; (e) Interferon-gamma production from the same challenge; and (f) Fold-proliferation from the same challenge.

FIG. 4. (a) Retroviral construct allowing 1:1 co-expression of CD34 marker gene with CAR; (b) Flow cytometric analysis of CAR expression (HA tag) vs CD34 marker gene; (c) Chromium release assay of non-transduced T-cells and T-cells transduced with the 3 different CAR variants against GD2 positive targets (LAN-1), and GD2 negative targets (A204); (d) Interferon gamma release; (e) IL-2 release; and (f) proliferation of the same targets and effectors.

FIG. 5—Introduction of FcR binding disrupting mutations into the Fc spacer (a) mutations introduced; (b) Expression of CAR as determined by anti-Fc staining: non-transduced, wt and mutated; (c) Killing of GD2 negative and GD2 positive targets with either non-transduced, wt Fc and mutated Fc anti-GD2 CAR T-cells; (d) Activation of non-transduced, wt Fc and mutated Fc anti-GD2 T-cells with the FcR expressing cell line THP-1; IL-1Beta release by THP-1 cell line in response to non-transduced, wt Fc and mutated Fc CAR T-cells.

FIG. 6—Optimization of the Expression Cassette
(a) map optimizations which were introduced into the cassette: SAR or CHS4; (b) representative expression of CAR with different modifications with either wt or codon-optimized open-reading frame. The SAR construct gives a tight peak of expression which is what is desired. (c) Bar chart representation of this FACS data from 3 normal donors.

FIG. 7—Comparison of different endodomains

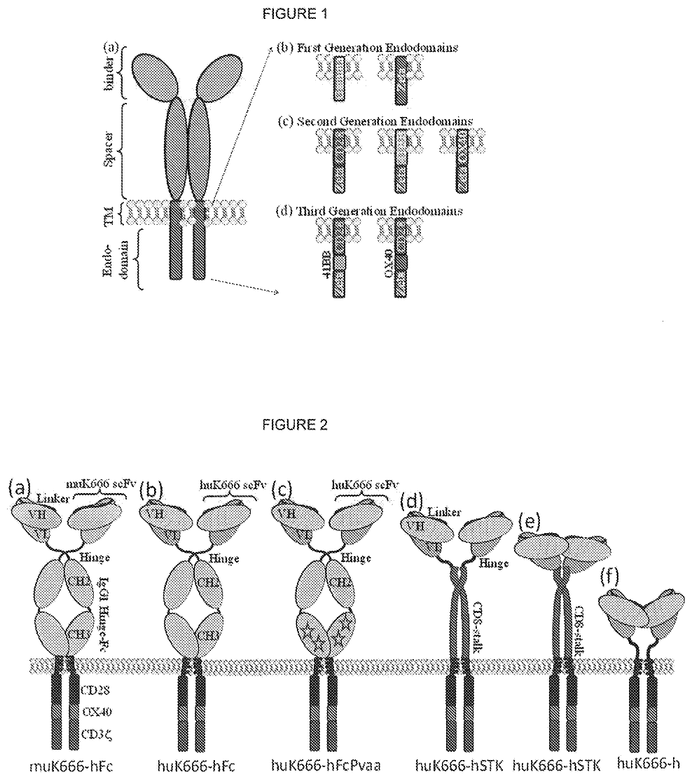

Three different chimeric antigen receptors were compared. The receptors all comprised of the huK666 scFv, the Fc domain of IgG1 mutated to reduce FcR binding and the CD28 transmembrane domain. CAR “28tmZ” has a CD3 Zeta endodomain; “28Z” has a compound CD28-CD3Zeta endodomain; “280XZ” has a compound endodomain comprising of CD28, OX40 and CD3Zeta, Peripheral blood T-cells from normal donors were transduced with these constructs with retroviral vectors of similar titers. These different T-cell lines were compared, along with non-transduced T-cells as controls. T-cells were challenged with A204 cells (a rhabdomyosarcoma cell line which is GD2 negative), and LAN-1 cells (a neuroblastoma cell line which is GD2 positive). Proliferation and cytokine release show that receptor activity is 28tmZ<28Z<28OXZ.

FIG. 8—Co-expression with iCasp9 suicide gene
(a) Co-expression of iCasp9 with anti-GD2CAR using FMD-2A sequence; (b) CAR expression in NT T-cells, GD2CAR transduced T-cells and iCasp9-2A-GD2CAR T-cells alone and after treatment with CID; (c) Killing of GD2 positive (LAN-1) and negative (A204) targets with non-transduced, GD2CAR transduced and iCasp9-2A-GD2CAR transduced T-cells with or without treatment with CID. Average of 5 normal donor T-cells.

FIG. 9—Co-expression with RQR8 suicide gene
(a) CARhuK666Fc was co-expressed with the RQR8 sort-suicide gene in a retroviral vector. (b) T-cells were transduced with this retroviral vector and co-expression of the CAR and RQR8 was determined by staining the transduced T-cells with a polyclonal anti-Fc and the monoclonal antibody QBend10. (c) The CAR positive population from these T-cells could be depleted in the presence of Rituximab and complement. (d) T-cells depleted with Rituximab no longer recognized GD2 expressing targets.

FIG. 10—(a) Bicistronic vector expressing GM3synthase and GD2synthase. (b) SupT1 cells transduced with this vector become GD2 positive (non-transduced empty plot; transduced greyed plot).

FIG. 11—Growth curves of individual tumours in mice in the following cohorts: top left: mice with GD2 expressing CT26 tumours receiving anti-GD2 CAR spleoncytes; top right: GD2 expressing CT26 tumours receiving mock-transduced splenocytes; bottom left: GD2 negative (wt) CT26 tumours with anti-GD2 CAR splenocytes; bottom right; and GD2 expressing CT26 tumours receiving no splenocytes

FIG. 12—Amino Acid Sequences
A. anti-GD2 CAR shown as (a) in FIG. 2 (muKM666-HCH2CH3-CD28OXZ—SEQ ID No. 26)
B. anti-GD2 CAR shown as (b) in FIG. 2 (huKM666-HCH2CH3-CD28OXZ—SEQ ID No. 27)
C. anti-GD2 CAR shown as (c) in FIG. 2 (huKM666-HCH2CH3pvaa-CD28OXZ—SEQ ID No. 28)
D. anti-GD2 CAR shown as (d) in FIG. 2 (huKM666-HSTK-CD28OXZ—SEQ ID No. 29)
E. anti-GD2 CAR shown as (e) in FIG. 2 (huKM666-STK-CD28XOXZ—SEQ ID No. 30)
F. anti-GD2 CAR shown as (f) in FIG. 2 (huKM666-HNG-CD28OXZ—SEQ ID No. 31)
G. anti-GD2 CAR as shown in (c) FIG. 2 but with 1st generation endodomain (huKM666-HCH2CH3pvaa-CD28tmZ—SEQ ID No. 32)
H. anti-GD2 CAR as shown in (c) FIG. 2 but with 2nd generation endodomain (huMK666-HCH2CH3pvaa-CD28Z—SEQ ID No. 33)
I. anti-GD2 CAR co-expressed with iCasp9 suicide gene—SEQ ID No. 34
J. anti-GD2 CAR co-expressed with RQR8 suicide gene—SEQ ID No. 35

FIG. 13—Structure of GD2

FIG. 14—Comparison of huk666 and 14g2a CARs. (a) maps of constructs tested: Two constructs were tested in primary T-cells. Both are retroviral vectors coding for RQR8 and a 2nd generation GD2 CAR co-expressed with a FMD-2A like sequence. The only difference between constructs is that in one, the scFv is huk666 and in the other it is 14g2a. T-cells transduced with these constructs were challenged 1:1 with either A204 (a GD2 negative rhabdomyosarcoma cell line), and LAN-1 (a GD2 positive cell line). (b) At 24 hours, Interferon-gamma was measured from supernatant. huK666 CAR T-cells produce more IF-G. (c) After one week T-cells are counted, huK666 show more proliferation.

FIG. 15—Flow cytometric analysis of co-culture between huk666 or 14g2a based 2nd generation CARs and Neuroblastoma cell line LAN1. (a) Set up of the experiment. After one week co-culture, cells were harvested and analyzed by flow-cytometry. CD45 expression allowed discrimination from lymphoid cells and non-lymphoid cells with CD45-cells being LAN-1 cells. Further staining with CD3/QBEND/10 allowed counting of CAR T-cells. (b) T-cells alone; (c) NT T-cells and LAN-1 cells; (d) huK666-28-Z CAR T-cells and LAN-1 cells; (e) 14g2a-28-Z CAR T-cells and LAN-1 cells. A residuum of LAN-1 cells is seen in the 14g2a CAR T-cell co-culture.

### SUMMARY OF ASPECTS OF THE INVENTION

The present inventors have constructed a new chimeric antigen receptor (CAR) targeting GD2 which comprises a GD2-binding domain based on the K666 antibody.

The anti-GD2 antibody 14g2a can be seen as the gold standard because it is used as a therapeutic antibody and is the only scFv tested to date in a CAR study (PMID: 18978797). The present inventors compared 14g2a and huk666 based CAR in a second generation format as this is the most widely used CAR format used in clinical studies. We found that huk666 CAR T-cells release more IFN-γ, proliferate better and kill more completely than 14g2a equivalents.

Thus, in a first aspect the present invention provides a chimeric antigen receptor (CAR) comprising a disialoganglioside (GD2)-binding domain which comprises
a) a heavy chain variable region (VH) having complementarity determining regions (CDRs) with the following sequences:

CDR1
(SEQ ID No. 1)
SYNIH;
CDR2
(SEQ ID No. 2)
VIWAGGSTNYNSALMS
CDR3
(SEQ ID No. 3)
RSDDYSWFAY;

and
b) a light chain variable region (VL) having CDRs with the following sequences:

CDR1
(SEQ ID No. 4)
RASSSVSSSYLH;
CDR2
(SEQ ID No. 5)
STSNLAS
CDR3
(SEQ ID No. 6)
QQYSGYPIT.

The GD2 binding domain may comprise a VH domain having the sequence shown as SEQ ID No. 9, or SEQ ID NO 10; or a VL domain having the sequence shown as SEQ ID No 11, or SEQ ID No. 12 or a variant thereof having at least 90% sequence identity which retains the capacity to i) bind GD2 and ii) induce T cell signalling.

The GD2 binding domain may comprise the sequence shown as SEQ ID No 7 or SEQ ID No. 8 or a variant thereof having at least 90% sequence identity which retains the capacity to i) bind GD2 and ii) induce T cell signalling.

The transmembrane domain may comprise the sequence shown as SEQ ID No. 13 or a variant thereof having at least 90% sequence identity which retains the capacity to i) bind GD2 and ii) induce T cell signalling.

The GD2-binding domain and the transmembrane domain may be connected by a spacer.

The spacer may comprise one of the following: a human an IgG1 Fc domain; an IgG1 hinge; an IgG1 hinge-CD8 stalk; or a CD8 stalk.

The spacer may comprise an IgG1 hinge-CD8 stalk or a CD8 stalk.

The spacer may comprise an IgG1 Fc domain or a variant thereof.

The spacer may comprise an IgG1 Fc domain which comprises the sequence shown as SEQ ID No. 23 or SEQ ID No. 24 or a variant thereof having at least 80% sequence identity.

The CAR may comprise or associate with an intracellular T cell signalling domain.

The intracellular T cell signalling domain may comprise one or more of the following endodomains: CD28 endodomain; OX40 and CD3-Zeta endodomain.

The intracellular T cell signalling domain may comprise all of the following endodomains: CD28 endodomain; OX40 and CD3-Zeta endodomain.

The CAR may comprise the sequence shown as any of SEQ ID No. 26 to 35 or a variant thereof which has at least 80% sequence identity but retains the capacity to i) bind GD2 and ii) induce T cell signalling.

In a second aspect, the present invention provides a nucleic acid sequence which encodes a CAR according to the first aspect of the invention.

The nucleic acid sequence may be codon-optimised.

The nucleic acid sequence may comprise the sequence shown as SEQ ID No 25 or a variant thereof having at least 90% sequence identity.

The nucleic acid may also encode a suicide gene.

In a third aspect, the present invention provides a vector which comprises a nucleic acid sequence according to the second aspect of the invention.

In a fourth aspect, the present invention provides a cell which expresses a CAR according to the first aspect of the invention. The cell may be a cytolytic immune cell, such as a T cell or natural killer (NK) cell.

The cell may co-expresses a CAR according to the first aspect of the invention and a suicide gene.

The suicide gene may, for example, be iCasp9 or RQR8.

In a fifth aspect, the present invention provides a method for making a cell according to the fourth aspect of the invention, which comprises the step of introducing a nucleic acid according to the second aspect of the invention into a cell.

In a sixth aspect, the present invention provides a pharmaceutical composition which comprises a vector according to the third aspect of the invention or a cell according to the second aspect of the invention, together with a pharmaceutically acceptable carrier, diluent or excipient.

In a seventh aspect, the present invention provides a method for treating cancer which comprises the step of administering a vector according to the third aspect of the invention or a cell according to the fourth aspect of the invention to a subject.

The cancer may be neuroblastoma.

In an eighth aspect, the present invention provides a vector according to the third aspect of the invention or a cell according to the fourth aspect of the invention for use in treating a cancer.

In a ninth aspect, the present invention provides the use of according to the third aspect of the invention or a cell according to the fourth aspect of the invention in the manufacture of a medicament for treating cancer.

In a tenth aspect, the present invention provides a method for making a GD2-expressing cell which comprises the step of introducing a nucleic acid encoding GM3 synthase and a nucleic acid encoding GD2 synthase into a cell.

In an eleventh aspect, the present invention provides a GD2-expressing cell which comprises a heterologous nucleic acid encoding GM3 synthase and a heterologous nucleic acid encoding GD2 synthase.

In an twelfth aspect, the present invention provides method for stimulating a cell according to the fourth aspect of the invention in vitro, which comprises the step of bringing the cell into contact with a GD2-expressing cell according to the eleventh aspect of the invention.

In a thirteenth aspect, the present invention provides an expression cassette expressing a CAR which comprises a scaffold attachment region (SAR).

The expression cassette may express a CAR according to the first aspect of the invention.

### DETAILED DESCRIPTION

### Chimeric Antigen Receptors (CARs)

Chimeric antigen receptors (CARs), also known as chimeric T cell receptors, artificial T cell receptors and chimeric immunoreceptors, are engineered receptors, which graft an arbitrary specificity onto an immune effector cell. In a classical CAR, the specificity of a monoclonal antibody is grafted on to a T cell. CAR-encoding nucleic acids may be transferred to T cells using, for example, retroviral vectors. In this way, a large number of cancer-specific T cells can be generated for adoptive cell transfer. Phase I clinical studies of this approach show efficacy.

The target-antigen binding domain of a CAR is commonly fused via a spacer and transmembrane domain to a signaling endodomain. When the CAR binds the target-antigen, this results in the transmission of an activating signal to the T-cell it is expressed on.

The CAR of the present invention comprises a GD2 binding domain which is based on the KM666 monoclonal antibody (Nakamura et al (2001) Cancer Immunol. Immunother. 50:275-284).

The CAR of the present invention comprises a GD2-binding domain which comprises
a) a heavy chain variable region (VH) having complementarity determining regions (CDRs) with the following sequences:

CDR1
(SEQ ID No. 1)
SYNIH;
CDR2
(SEQ ID No. 2)
VIWAGGSTNYNSALMS
CDR3
(SEQ ID No. 3)
RSDDYSWFAY;

and
b) a light chain variable region (VL) having CDRs with the following sequences:

CDR1
(SEQ ID No. 4)
RASSSVSSSYLH;
CDR2
(SEQ ID No. 5)
STSNLAS
CDR3
(SEQ ID No. 6)
QQYSGYPIT.

It may be possible to introduce one or more mutations (substitutions, additions or deletions) into the or each CDR without negatively affecting GD2-binding activity. Each CDR may, for example, have one, two or three amino acid mutations.

The CAR of the present invention may comprise one of the following amino acid sequences:

(Murine KM666 sequence)
SEQ ID No. 7
QVQLKESGPVLVAPSQTLSITCTVSGFSLASYNIHWVRQPPGKGLEWLGV
IWAGGSTNYNSALMSRLSISKDNSKSQVFLQMNSLQTDDTAMYYCAKRSD
DYSWFAYWGQGTLVTVSASGGGGSGGGGSGGGGSENVLTQSPAIMSASPG
EKVTMTCRASSSVSSSYLHWYQQKSGASPKVWIYSTSNLASGVPGRFSGS
GSGTSYSLTISSVEAEDAATYYCQQYSGYPITFGAGTKVEVKR
(Humanised KM666 sequence)
SEQ ID No. 8
QVQLQESGPGLVKPSQTLSITCTVSGFSLASYNIHWVRQPPGKGLEWLGV
IWAGGSTNYNSALMSRLTISKDNSKNQVFLKMSSLTAADTAVYYCAKRSD
DYSWFAYWGQGTLVTVSSGGGGSGGGGSGGGGSENQMTQSPSSLSASVGD
RVTMTCRASSSVSSSYLHWYQQKSGKAPKVWIYSTSNLASGVPSRFSGSG
SGTDYTLTISSLQPEDFATYYCQQYSGYPITFGQGTKVEIKR

The CAR of the present invention may comprise one of the following VH sequences:

(Murine KM666 VH sequence)
SEQ ID No. 9
QVQLKESGPVLVAPSQTLSITCTVSGFSLASYNIHWVRQPPGKGLEWLGV
IWAGGSTNYNSALMSRLSISKDNSKSQVFLQMNSLQTDDTAMYYCAKRSD
DYSWFAYWGQGTLVTVSA
(Humanised KM666 VH sequence)
SEQ ID No. 10
QVQLQESGPGLVKPSQTLSITCTVSGFSLASYNIHWVRQPPGKGLEWLGV
IWAGGSTNYNSALMSRLTISKDNSKNQVFLKMSSLTAADTAVYYCAKRSD
DYSWFAYWGQGTLVTVSS

The CAR of the present invention may comprise one of the following VL sequences:

(Murine KM666 VL sequence)
SEQ ID No. 11
ENVLTQSPAIMSASPGEKVTMTCRASSSVSSSYLHWYQQKSGASPKVWIY
STSNLASGVPGRFSGSGSGTSYSLTISSVEAEDAATYYCQQYSGYPITFG
AGTKVEVK
(Humanised KM666 VH sequence)
SEQ ID No. 12
ENQMTQSPSSLSASVGDRVTMTCRASSSVSSSYLHWYQQKSGKAPKVWIY
STSNLASGVPSRFSGSGSGTDYTLTISSLQPEDFATYYCQQYSGYPITFG
QGTKVEIK

The CAR of the invention may comprise a variant of the sequence shown as SEQ ID No. 7, 8, 9, 10, 11 or 12 having at least 80, 85, 90, 95, 98 or 99% sequence identity, provided that the variant sequence retain the capacity to bind GD2 (when in conjunction with a complementary VL or VH domain, if appropriate).

The percentage identity between two polypeptide sequences may be readily determined by programs such as BLAST which is freely available at http://blast.ncbi.nlm.nih.gov.

### Transmembrane Domain

The CAR of the invention may also comprise a transmembrane domain which spans the membrane. It may comprise a hydrophobic alpha helix. The transmembrane domain may be derived from CD28, which gives good receptor stability.

The transmembrane domain may comprise the sequence shown as SEQ ID No. 13.

SEQ ID No. 13
FWVLVVVGGVLACYSLLVTVAFIIFWV

### Intracellular T Cell Signaling Domain (Endodomain)

The endodomain is the signal-transmission portion of the CAR. After antigen recognition, receptors cluster and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

The endodomain of the CAR of the present invention may comprise the CD28 endodomain and OX40 and CD3-Zeta endodomain.

The transmembrane and intracellular T-cell signalling domain (endodomain) of the CAR of the present invention may comprise the sequence shown as SEQ ID No. 14, 15, 16, 17 or 18 or a variant thereof having at least 80% sequence identity.

(CD28 endodomain)
SEQ ID No. 14
RSKRSRLLHSDYMNMTPRRPGPTRKHYQPYAPPRDFAAY
(CD40 endodomain)
SEQ ID No. 15
RSRDQRLPPDAHKPPGGGSFRTPIQEEQADAHSTLAKI
(CD3 zeta endodomain)
SEQ ID No. 16
RSRVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTATK
DTYDALHMQALPPR
(CD28Z)
SEQ ID No. 17
RSKRSRLLHSDYMNMTPRRPGPTRKHYQPYAPPRDFAAYRSRVKFSRSAD
APAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQAL
PPR
(CD28OXZ)
SEQ ID No. 18
RSKRSRLLHSDYMNMTPRRPGPTRKHYQPYAPPRDFAAYRSRDQRLPPDA
HKPPGGGSFRTPIQEEQADAHSTLAKIRVKFSRSADAPAYQQGQNQLYNE
LNLGRREEYDVLDKRRGRDPEMGGKPRRKNPQEGLYNELQKDKMAEAYSE
IGMKGERRRGKGHDGLYQGLSTATKDTYDALHMQALPPR

A variant sequence may have at least 80%, 85%, 90%, 95%, 98% or 99% sequence identity to SEQ ID No. 13, 14, 15, 16, 17 or 18, provided that the sequence provides an effective transmembrane domain/intracellular T cell signaling domain.

### Signal Peptide

The CAR of the present invention may comprise a signal peptide so that when the CAR is expressed inside a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The CAR of the invention may have the general formula:

Signal peptide—GD2-binding domain—spacer domain—transmembrane domain—intracellular T cell signaling domain.

The signal peptide may comprise the SEQ ID No. 19 or a variant thereof having 5, 4, 3, 2 or 1 amino acid mutations (insertions, substitutions or additions) provided that the signal peptide still functions to cause cell surface expression of the CAR.

SEQ ID No. 19:
METDTLLLWVLLLWVPGSTG

The signal peptide of SEQ ID No. 19 is compact and highly efficient. It is predicted to give about 95% cleavage after the terminal glycine, giving efficient removal by signal peptidase.

### Spacer

The CAR of the present invention may comprise a spacer sequence to connect the GD2-binding domain with the transmembrane domain and spatially separate the GD2-binding domain from the endodomain. A flexible spacer allows to the GD2-binding domain to orient in different directions to enable GD2 binding.

The spacer sequence may, for example, comprise an IgG1 Fc region, an IgG1 hinge or a CD8 stalk, or a combination thereof. The spacer may alternatively comprise an alternative sequence which has similar length and/or domain spacing properties as an IgG1 Fc region, an IgG1 hinge or a CD8 stalk.

A human IgG1 spacer may be altered to remove Fc binding motifs.

Examples of amino acid sequences for these spacers are given below:

(hinge-CH2CH3 of human IgG1)
SEQ ID No. 20
AEPKSPDKTHTCPPCPAPPVAGPSVFLFPPKPKDTLMIARTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGKKD
(human CD8 stalk):
SEQ ID No. 21
TTTPAPRPPTPAPTIASQPLSLRPEACRPAAGGAVHTRGLDFACD1
(human IgG1 hinge):
SEQ ID No. 22
AEPKSPDKTHTCPPCPKDPK
(IgG1 Hinge-Fc)
SEQ ID No. 23
AEPKSPDKTHTCPPCPAPELLGGPSVFLFPPKPKDILMISRTPEVTCVVV
DVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDK
SRWQQGNVFSCSVMHEALHNHYTQKSLSLSPGKKDPK
(IgG1 Hinge-Fc modified to remove Fc receptor
recognition motifs)
SEQ ID No. 24
AEPKSPDKTHTCPPCPAPPVA*GPSVFLFPPKPKDTLMIARTPEVTCVVV
DVSHEDPEVKFNWYVDGVEVHNAKTKPREEQYNSTYRVVSVUTVLHQDWL
NGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDK
SRWQQGNVFSCSVMHEALHNHYTQKSLSLSPGKKDPK

Modified residues are underlined; * denotes a deletion.

### GD2

GD2 is a disialoganglioside expressed on tumors of neuroectodermal origin, including human neuroblastoma and melanoma, with highly restricted expression on normal tissues, principally to the cerebellum and peripheral nerves in humans.

The relatively tumour specific expression of GD2 makes it a suitable target for immunotherapy.

### Nucleic Acid Sequence

The second aspect of the invention relates to a nucleic acid sequence which codes for a CAR of the first aspect of the invention.

The nucleic acid sequence may be capable of encoding a CAR having the amino acid sequence shown as any of SEQ ID No. 26-35.

The nucleic acid sequence may be or comprise the following sequence:

| SEQ ID No. 25 DNA sequence of retroviral cassette comprising of anti-GD2 CAR | |
|---|---|
| co-expressed with RQR8 suicide gene with a codon-optimized frame and a SAR | |
| region to enhance expression | |
| 1 | tgaaagaccc cacctgtagg tttggcaagc tagcttaagt aacgccattt tgcaaggcat ggaaaaatac |
| | >>...................................LTR...................................> |
| 71 | ataactgaga atagaaaagt tcagatcaag gtcaggaaca gatggaacag ctgaatatgg gccaaacagg |
| | >...................................LTR....................................> |
| 141 | atatctgtgg taagcagttc ctgccccggc tcagggccaa gaacagatgg aacagctgaa tatgggccaa |
| | >...................................LTR....................................> |
| 211 | acaggatatc tgtggtaagc agttcctgcc ccggctcagg gccaagaaca gatggtcccc agatgcggtc |
| | >...................................LTR....................................> |
| 281 | cagccctcag cagtttctag agaaccatca gatgtttcca gggtgcccca aggacctgaa atgaccctgt |
| | >...................................LTR....................................> |
| 351 | gccttatttg aactaaccaa tcagttcgct tctcgcttct gttcgcgcgc ttatgctccc cgagctcaat |
| | >...................................LTR....................................> |
| 421 | aaaagagccc acaacccctc actcggggcg ccagtcctcc gattgactga gtcgcccggg tacccgtgta |
| | >...................................LTR....................................> |
| 491 | tccaataaac cctcttgcag ttgcatccga cttgtggtct cgctgttcct tgggagggtc tcctctgagt |
| | >...................................LTR....................................> |
| 561 | gattgactac ccgtcagcgg gggtctttca tttgggggct cgtccgggat cgggagaccc ctgcccaggg |
| | >.............LTR.............>> |
| 631 | accaccgacc caccaccggg aggtaagctg gccagcaact tatctgtgtc tgtccgattg tctagtgtct |
| 701 | atgactgatt ttatgcgcct gcgtcggtac tagttagcta actagctctg tatctggcgg acccgtggtg |
| | Eco52I |
| | ------ |
| 771 | gaactgacga gttcggaaca cccggccgca accctgggag acgtcccagg gacttcgggg gccgtttttg |
| | PshAI |
| | ----------- |
| 841 | tggcccgacc tgagtcctaa aatcccgatc gtttaggact ctttggtgca ccccccttag aggagggata |
| 911 | tgtggttctg gtaggagacg agaacctaaa acagttcccg cctccgtctg aatttttgct ttcggtttgg |
| 981 | gaccgaagcc gcgccgcgcg tcttgtctgc tgcagcatcg ttctgtgttg tctctgtctg actgtgtttc |
| | SrfI |
| | -------- |
| 1051 | tgtatttgtc tgaaaatatg ggcccgggct agcctgttac cactccctta agtttgacct taggtcactg |
| 1121 | gaaagatgtc gagcggatcg ctcacaacca gtcggtagat gtcaagaaga gacgttgggt taccttctgc |
| 1191 | tctgcagaat ggccaacctt taacgtcgga tggccgcgag acggcacctt taaccgagac ctcatcaccc |
| 1261 | aggttaagat caaggtcttt tcacctggcc cgcatggaca cccagaccag gtggggtaca tcgtgacctg |
| 1331 | ggaagccttg gcttttgacc cccctccctg ggtcaagccc tttgtacacc ctaagcctcc gcctcctctt |
| 1401 | cctccatccg ccccgtctct cccccttgaa cctcctcgtt cgaccccgcc tcgatcctcc ctttatccag |
| | BglII |
| | ------- |
| 1471 | ccctcactcc ttctctaggc gcccccatat ggccatatga gatcttatat ggggcacccc cgccccttgt |
| 1541 | aaacttccct gaccctgaca tgacaagagt tactaacagc ccctctctcc aagctcactt acaggctctc |
| | AgeI |
| | ------ |
| 1611 | tacttagtcc agcacgaagt ctggagacct ctggcggcag cctaccaaga acaactggac cgaccggtgg |
| 1681 | tacctcaccc ttaccgagtc ggcgacacag tgtgggtccg ccgacaccag actaagaacc tagaacctcg |
| | AccI |
| | ------- |
| 1751 | ctggaaagga ccttacacag tcctgctgac cacccccacc gccctcaaag tagacggcat cgcagcttgg |
| | PmlI |
| | ------ |
| 1821 | atacacgccg cccacgtgaa ggctgccgac cccgggggtg gaccatcctc tagactgcca acatgggcac |
| | >>.orf.> |
| | >>RQR8.> |
| 1891 | cagcctgctg tgctggatgg ccctgtgcct gctgggcgcc gaccacgccg atgcctgccc ctacagcaac |
| | >...................................orf....................................> |
| | >...................................RQR8...................................> |
| 1961 | cccagcctgt gcagcggagg cggcggcagc gagctgccca cccagggcac cttctccaac gtgtccacca |
| | >...................................orf....................................> |
| | >...................................RQR8...................................> |
| 2031 | acgtgagccc agccaagccc accaccaccg cctgtcctta ttccaatcct tccctgtgta gcggaggggg |
| | >...................................orf....................................> |
| | >...................................RQR8...................................> |
| 2101 | aggcagccca gcccccagac ctcccacccc agcccccacc atcgccagcc agcctctgag cctgagaccc |
| | >...................................orf....................................> |
| | >...................................RQR8...................................> |
| | SgrAI |
| | --------- |
| 2171 | gaggcctgcc gcccagccgc cggcggcgcc gtgcacacca gaggcctgga tttcgcctgc gatatctaca |
| | >...................................orf....................................> |
| | >...................................RQR8...................................> |
| | BclI |
| | ------- |
| 2241 | tctgggcccc actggccggc acctgtggcg tgctgctgct gagcctggtg atcaccctgt actgcaacca |
| | >...................................orf....................................> |
| | >...................................RQR8...................................> |
| 2311 | ccgcaaccgc aggcgcgtgt gcaagtgccc caggcccgtg gtgagagccg agggcagagg cagcctgctg |
| | >...................................orf....................................> |
| | >....................RQR8....................>> |
| | >>..........FMD-2A..........> |
| | NcoI |
| | ------ |
| 2381 | acctgcggcg acgtggagga gaacccaggc cccatggaga ccgacaccct gctgctgtgg gtgctgctgc |
| | >...................................orf....................................> |
| | >.............FMD-2A..............>> |
| | >>.................CAR.................> |
| 2451 | tgtgggtgcc aggcagcacc ggccaggtgc agctgcagga gtctggccca ggcctggtga agcccagcca |
| | >...................................orf....................................> |
| | >...................................CAR....................................> |
| 2521 | gaccctgagc atcacctgca ccgtgagcgg cttcagcctg gccagctaca acatccactg ggtgcggcag |
| | >...................................orf....................................> |
| | >...................................CAR....................................> |
| 2591 | cccccaggca agggcctgga gtggctgggc gtgatctggg ctggcggcag caccaactac aacagcgccc |
| | >...................................orf....................................> |
| | >...................................CAR....................................> |
| 2661 | tgatgagccg gctgaccatc agcaaggaca acagcaagaa ccaggtgttc ctgaagatga gcagcctgac |
| | >...................................orf....................................> |
| | >...................................CAR....................................> |
| 2731 | agccgccgac accgccgtgt actactgcgc caagcggagc gacgactaca gctggttcgc ctactggggc |
| | >...................................orf....................................> |
| | >...................................CAR....................................> |
| 2801 | cagggcaccc tggtgaccgt gagctctggc ggaggcggct ctggcggagg cggctctggc ggaggcggca |
| | >...................................orf....................................> |
| | >...................................CAR....................................> |
| 2871 | gcgagaacca gatgacccag agccccagca gcttgagcgc cagcgtgggc gaccgggtga ccatgacctg |
| | >...................................orf....................................> |
| | >...................................CAR....................................> |
| 2941 | cagagccagc agcagcgtga gcagcagcta cctgcactgg taccagcaga agagcggcaa ggccccaaag |
| | >...................................orf....................................> |
| | >...................................CAR....................................> |
| 3011 | gtgtggatct acagcaccag caacctggcc agcggcgtgc ccagccggtt cagcggcagc ggcagcggca |
| | >...................................orf....................................> |
| | >...................................CAR....................................> |
| 3081 | ccgactacac cctgaccatc agcagcctgc agcccgagga cttcgccacc tactactgcc agcagtacag |
| | >...................................orf....................................> |
| | >...................................CAR....................................> |
| | BamHI |
| | ------ |
| 3151 | cggctacccc atcaccttcg gccagggcac caaggtggag atcaagcggt cggatcccgc cgagcccaaa |
| | >...................................orf....................................> |
| | >...................................CAR....................................> |
| | FseI |
| | --------- |
| 3221 | tctcctgaca aaactcacac atgcccaccg tgcccagcac ctcccgtggc cggcccgtca gtcttcctct |
| | >...................................orf....................................> |
| | >...................................CAR....................................> |
| 3291 | tccccccaaa acccaaggac accctcatga tcgcccggac ccctgaggtc acatgcgtgg tggtggacgt |
| | >...................................orf....................................> |
| | >...................................CAR....................................> |
| 3361 | gagccacgaa gaccctgagg tcaagttcaa ctggtacgtg gacggcgtgg aggtgcataa tgccaagaca |
| | >...................................orf....................................> |
| | >...................................CAR....................................> |
| | SacII |
| | ------ |
| 3431 | aagccgcggg aggagcagta caacagcacg taccgtgtgg tcagcgtcct caccgtcctg caccaggact |
| | >...................................orf....................................> |
| | >...................................CAR....................................> |
| 3501 | ggctgaatgg caaggagtac aagtgcaagg tctccaacaa agccctccca gcccccatcg agaaaaccat |
| | >...................................orf....................................> |
| | >...................................CAR....................................> |
| 3571 | ctccaaagcc aaagggcagc cccgagaacc acaggtgtac accctgcccc catcccggga tgagctgacc |
| | >...................................orf....................................> |
| | >...................................CAR....................................> |
| 3641 | aagaaccagg tcagcctgac ctgcctggtc aaaggcttct atcccagcga catcgccgtg gagtgggaga |
| | >...................................orf....................................> |
| | >...................................CAR....................................> |
| 3711 | gcaatgggca accggagaac aactacaaga ccacgcctcc cgtgctggac tccgacggct ccttcttcct |
| | >...................................orf....................................> |
| | >...................................CAR....................................> |
| | Ppu10I |
| | ------ |
| | NsiI |
| | ------ |
| | BfrBI |
| | ------ |
| 3781 | ctacagcaag ctcaccgtgg acaagagcag gtggcagcag gggaacgtct tctcatgctc cgtgatgcat |
| | >...................................orf....................................> |
| | >...................................CAR....................................> |
| | Van91I |
| | ----------- |
| 3851 | gaggccctgc acaatcacta tacccagaaa tctctgagtc tgagcccagg caagaaggac cccaagttct |
| | >...................................orf....................................> |
| | >...................................CAR....................................> |
| 3921 | gggtcctggt ggtggtggga ggcgtgctgg cctgttactc tctcctggtg accgtggcct tcatcatctt |
| | >...................................orf....................................> |
| | >...................................CAR....................................> |
| 3991 | ctgggtgcgc tccaagagga gcaggctcct gcacagtgac tacatgaaca tgactccccg ccgccccggg |
| | >...................................orf....................................> |
| | >...................................CAR....................................> |
| 4061 | cccacccgca agcattacca gccctatgcc ccaccacgcg acttcgcagc ctatcgctcc cgggtgaagt |
| | >...................................orf....................................> |
| | >...................................CAR....................................> |
| 4131 | tctctcgctc tgccgatgcc ccagcctatc agcagggcca gaatcagctg tacaatgaac tgaacctggg |
| | >...................................orf....................................> |
| | >...................................CAR....................................> |
| 4201 | caggcgggag gagtacgacg tgctggataa gcggagaggc agagaccccg agatgggcgg caaaccacgg |
| | >...................................orf....................................> |
| | >...................................CAR....................................> |
| 4271 | cgcaaaaatc cccaggaggg actctataac gagctgcaga aggacaaaat ggccgaggcc tattccgaga |
| | >...................................orf....................................> |
| | >...................................CAR....................................> |
| 4341 | tcggcatgaa gggagagaga agacgcggaa agggccacga cggcctgtat cagggattgt ccaccgctac |
| | >...................................orf....................................> |
| | >...................................CAR....................................> |
| | MluI ClaI |
| | ------------- |
| 4411 | aaaagataca tatgatgccc tgcacatgca ggccctgcca cccagatgac gcgtatcgat actgttctca |
| | >.......................orf........................>> |
| | >.......................CAR........................>> |
| | >>..SAR..> |
| 4481 | tcacatcata tcaaggttat ataccatcaa tattgccaca gatgttactt agccttttaa tatttctcta |
| | >...................................SAR....................................> |
| 4551 | atttagtgta tatgcaatga tagttctctg atttctgaga ttgagtttct catgtgtaat gattatttag |
| | >...................................SAR....................................> |
| 4621 | agtttctctt tcatctgttc aaatttttgt ctagttttat tttttactga tttgtaagac ttctttttat |
| | >...................................SAR....................................> |
| 4691 | aatctgcata ttacaattct ctttactggg gtgttgcaaa tattttctgt cattctatgg cctgactttt |
| | >...................................SAR....................................> |
| 4761 | cttaatggtt ttttaatttt aaaaataagt cttaatattc atgcaatcta attaacaatc ttttctttgt |
| | >...................................SAR....................................> |
| | SphI |
| | ------ |
| 4831 | ggttaggact ttgagtcata agaaattttt ctctacactg aagtcatgat ggcatgcttc tatattattt |
| | >...................................SAR....................................> |
| 4901 | tctaaaagat ttaaagtttt gccttctcca tttagactta taattcactg gaattttttt gtgtgtatgg |
| | >...................................SAR....................................> |
| 4971 | tatgacatat gggttccctt ttatttttta catataaata tatttccctg tttttctaaa aaagaaaaag |
| | >...................................SAR....................................> |
| 5041 | atcatcattt tcccattgta aaatgccata tttttttcat aggtcactta catatatcaa tgggtctgtt |
| | >...................................SAR....................................> |
| 5111 | tctgagctct actctatttt atcagcctca ctgtctatcc ccacacatct catgctttgc tctaaatctt |
| | >...................................SAR....................................> |
| 5181 | gatatttagt ggaacattct ttcccatttt gttctacaag aatatttttg ttattgtctt tgggctttct |
| | >...................................SAR....................................> |
| 5251 | atatacattt tgaaatgagg ttgacaagtt cggattagtc caatttgtta aagacaggat atcagtggtc |
| | >..............SAR..............>> |
| 5321 | caggctctag ttttgactca acaatatcac cagctgaagc ctatagagta cgagccatag ataaaataaa |
| 5391 | agattttatt tagtctccag aaaaaggggg gaatgaaaga ccccacctgt aggtttggca agctagctta |
| | >>.................LTR.................> |
| 5461 | agtaacgcca ttttgcaagg catggaaaaa tacataactg agaatagaga agttcagatc aaggtcagga |
| | >...................................LTR....................................> |
| 5531 | acagatggaa cagctgaata tgggccaaac aggatatctg tggtaagcag ttcctgcccc ggctcagggc |
| | >...................................LTR....................................> |
| 5601 | caagaacaga tggaacagct gaatatgggc caaacaggat atctgtggta agcagttcct gccccggctc |
| | >...................................LTR....................................> |
| 5671 | agggccaaga acagatggtc cccagatgcg gtccagccct cagcagtttc tagagaacca tcagatgttt |
| | >...................................LTR....................................> |
| 5741 | ccagggtgcc ccaaggacct gaaatgaccc tgtgccttat ttgaactaac caatcagttc gcttctcgct |
| | >...................................LTR....................................> |
| 5811 | tctgttcgcg cgcttctgct ccccgagctc aataaaagag cccacaaccc ctcactcggg gcgccagtcc |
| | >...................................LTR....................................> |
| 5881 | tccgattgac tgagtcgccc gggtacccgt gtatccaata aaccctcttg cagttgcatc cgacttgtgg |
| | >...................................LTR....................................> |
| 5951 | tctcgctgtt ccttgggagg gtctcctctg agtgattgac tacccgtcag cgggggtctt tcac |
| | >...............................LTR................................>> |

The nucleic acid sequence may encode the same amino acid sequence as that encoded by SEQ ID No. 25, but may have a different nucleic acid sequence, due to 65 the degeneracy of the genetic code. The nucleic acid sequence may have at least 80, 85, 90, 95, 98 or 99% identity to the sequence shown as SEQ ID No. 25, provided that it encodes a CAR as defined in the first aspect of the invention.

### Suicide Genes

Since T-cells engraft and are autonomous, a means of selectively deleting CAR T-cells in recipients of anti-GD2 CAR T-cells is desirable. Suicide genes are genetically encodable mechanisms which result in selective destruction of infused T-cells in the face of unacceptable toxicity. The earliest clinical experience with suicide genes is with the Herpes Virus Thymidine Kinase (HSV-TK) which renders T-cells susceptible to Ganciclovir. HSV-TK is a highly effective suicide gene. However, pre-formed immune responses may restrict its use to clinical settings of considerable immunosuppression such as haploidentical stem cell transplantation. Inducible Caspase 9 (iCasp9) is a suicide gene constructed by replacing the activating domain of Caspase 9 with a modified FKBP12. iCasp9 is activated by an otherwise inert small molecular chemical inducer of dimerization (CID). iCasp9 has been recently tested in the setting of haploidentical HSCT and can abort GvHD. The biggest limitation of iCasp9 is dependence on availability of clinical grade proprietary CID. Both iCasp9 and HSV-TK are intracellular proteins, so when used as the sole transgene, they have been co-expressed with a marker gene to allow selection of transduced cells.

An iCasp9 may comprise the sequence shown as SEQ ID No. 36 or a variant thereof having at least 80, 90, 95 or 98% sequence identity.

SEQ ID No. 36
MLEGVQVETISPGDGRTFPKRGQTCVVHYTGMLEDGKKVDSSRDRNKPFK
FMLGKQEVIRGWEEGVAQMSVGQRAKLTISPDYAYGATGHPGIIPPHATL
VFDVELLKLESGGGSGVDGFGDVGALESLRGNADLAYILSMEPCGHCLII
NNVNFCRESGLRTRTGSNIDCEKLRRRFSSLHFMVEVKGDLTAKKMVLAL
LELAQQDHGALDCCVVVILSHGCQASHLQFPGAVYGTDGCPVSVEKIVNI
FNGTSCPSLGGKPKLFFIQACGGEQKDHGFEVASTSPEDESPGSNPEPDA
TPFQEGLRTFDQLDAISSLPTPSDIFVSYSTFPGFVSWRDPKSGSWYVET
LDDIFEQWAHSEDLQSLLLRVANAVSVKGIYKQMPGCFNFLRKKLFFKTS
AS

The present inventors have recently described a novel marker/suicide gene known as RQR8 which can be detected with the antibody QBEnd10 and expressing cells lysed with the therapeutic antibody Rituximab.

An RQR8 may comprise the sequence shown as SEQ ID No. 37 or a variant thereof having at least 80, 90, 95 or 98% sequence identity.

SEQ ID No. 37
MGTSLLCWMALCLLGADHADACPYSNPSLCSGGGGSELPTQGTFSNVSTN
VSPAKPTTTACPYSNPSLCSGGGGSPAPRPPTPAPTIASQPLSLRPEACR
PAAGGAVHTRGLDFACDIYIWAPLAGTCGVLLLSLVITLYCNHRNRRRVC
KCPRPVV

The suicide gene may be expressed as a single polypeptide with the CAR, for example by using a self-cleaving peptide between the two sequences.

### Vector

The present invention also provides a vector which comprises a nucleic acid sequence according to the present invention. Such a vector may be used to introduce the nucleic acid sequence into a host cell so that it expresses and produces a molecule according to the first aspect of the invention.

The vector may, for example, be a plasmid or a viral vector, such as a retroviral vector or a lentiviral vector.

The vector may be capable of transfecting or transducing a T cell.

The vector may also comprise a nucleic acid sequence encoding a suicide gene, such as iCasp9 or RQR8.

### Host Cell

The invention also provides a host cell which comprises a nucleic acid according to the invention. The host cell may be capable of expressing a CAR according to the first aspect of the invention.

The host cell may be a cytolytic immune cell such as a human T cell or natural killer (NK) cell.

A T-cell capable of expressing a CAR according to the invention may be made by transducing or transfecting a T cell with CAR-encoding nucleic acid.

The CAR T-cell may be generated ex vivo. The T cell may be from a peripheral blood mononuclear cell (PBMC) sample from the patient or a donor. T cells may be activated and/or expanded prior to being transduced with CAR-encoding nucleic acid, for example by treatment with an anti-CD3 monoclonal antibody.

### Pharmaceutical Composition

The present invention also relates to a pharmaceutical composition containing a vector or a CAR-expressing T cell of the invention together with a pharmaceutically acceptable carrier, diluent or excipient, and optionally one or more further pharmaceutically active polypeptides and/or compounds. Such a formulation may, for example, be in a form suitable for intravenous infusion).

### Method of Treatment

T cells expressing a CAR molecule of the present invention are capable of killing cancer cells, such as neurobastoma cells. CAR-expressing T cells may either be created ex vivo either from a patient's own peripheral blood (1st party), or in the setting of a haematopoietic stem cell transplant from donor peripheral blood (2nd party), or peripheral blood from an unconnected donor (3rd party). Alternatively, CAR T-cells may be derived from ex-vivo differentiation of inducible progenitor cells or embryonic progenitor cells to T-cells. In these instances, CAR T-cells are generated by introducing DNA or RNA coding for the CAR by one of many means including transduction with a viral vector, transfection with DNA or RNA.

T cells expressing a CAR molecule of the present invention may be used for the treatment of a cancerous disease, in particular a cancerous disease associated with GD2 expression.

The cancer may be an ectodermal tumour.

Examples of cancers which correlate with elevated GD2 expression levels are: neuroblastoma, melanoma, medulloblastoma, soft-tissue sarcomas, osteosarcoma and small-cell lung cancers such as NSCLC.

A method for the treatment of disease relates to the therapeutic use of a vector or T cell of the invention. In this respect, the vector or T cell may be administered to a subject having an existing disease or condition in order to lessen, reduce or improve at least one symptom associated with the disease and/or to slow down, reduce or block the progression of the disease. The method of the invention may cause or promote T-cell mediated killing of GD2-expressing cells, such as cancer cells.

### GD2 Expressing Cell

The invention also provides a method for making a GD2-expressing cell which comprises the step of introducing a nucleic acid encoding GM3 synthase and a nucleic acid encoding GD2 synthase into a cell.

The nucleic acid may be introduced by, for example, transfection or transduction, using a vector such as a plasmid or viral vector.

The invention also relates to a GD2-expressing cell which comprises a heterologous nucleic acid encoding GM3 synthase and a heterologous nucleic acid encoding GD2 synthase.

The nucleic acid may be “heterologous” in the sense that it is not usually present in the cell. It is an artificially introduced recombinant nucleic acid sequence.

The cell may be from a cell line.

The cell may be used for stimulating GD2CAR T-cells in culture, such as the T cells of the present invention.

The invention will now be further described by way of Examples, which are meant to serve to assist one of ordinary skill in the art in carrying out the invention and are not intended in any way to limit the scope of the invention.

### EXAMPLES

### Example 1—Using the Humanized Antibody Huk666 as a Binder

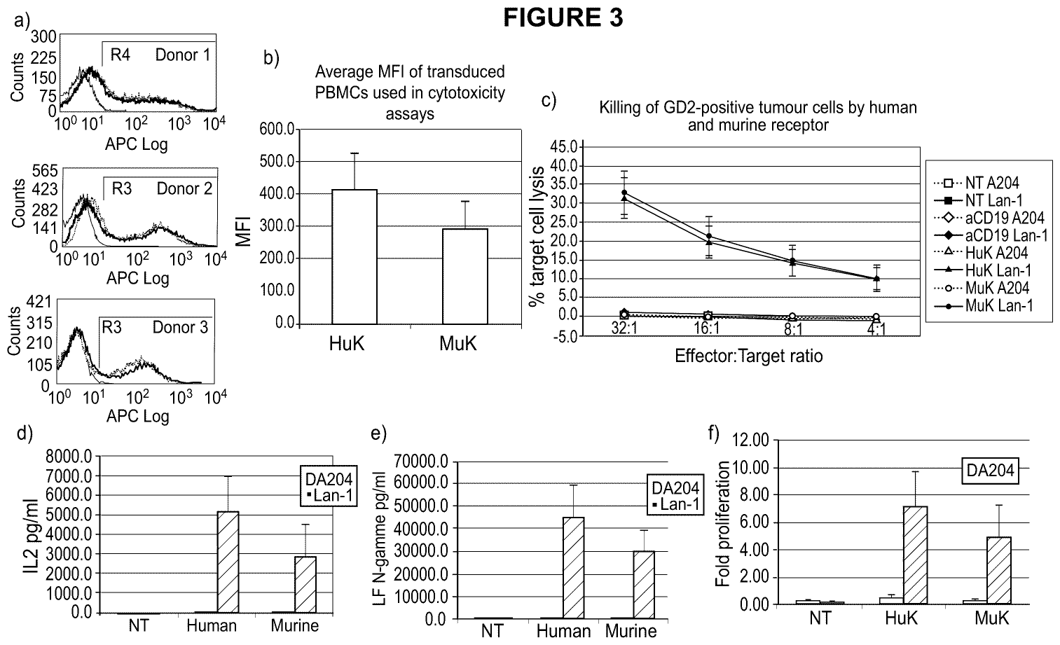

CARs were constructed with scFvs using sequences from either the mouse antibody KM666 or its humanized version huk666 as described by Nakamura et al (2001—as above) (variants (a) and (b) in FIG. 2 above). These receptors were compared for expression/stability and found to be equal for both receptors. Next, killing, cytokine release and proliferation of T-cells transduced with these receptors were tested when challenged by target cells either not expressing or expressing GD2. It was concluded that killing of both receptors was similar, but the humanized scFv based receptor resulted in superior IL2 production and proliferation (FIG. 3).

### Example 2—Testing the Effect of Different Spacer Formats Effects on Expression and Function

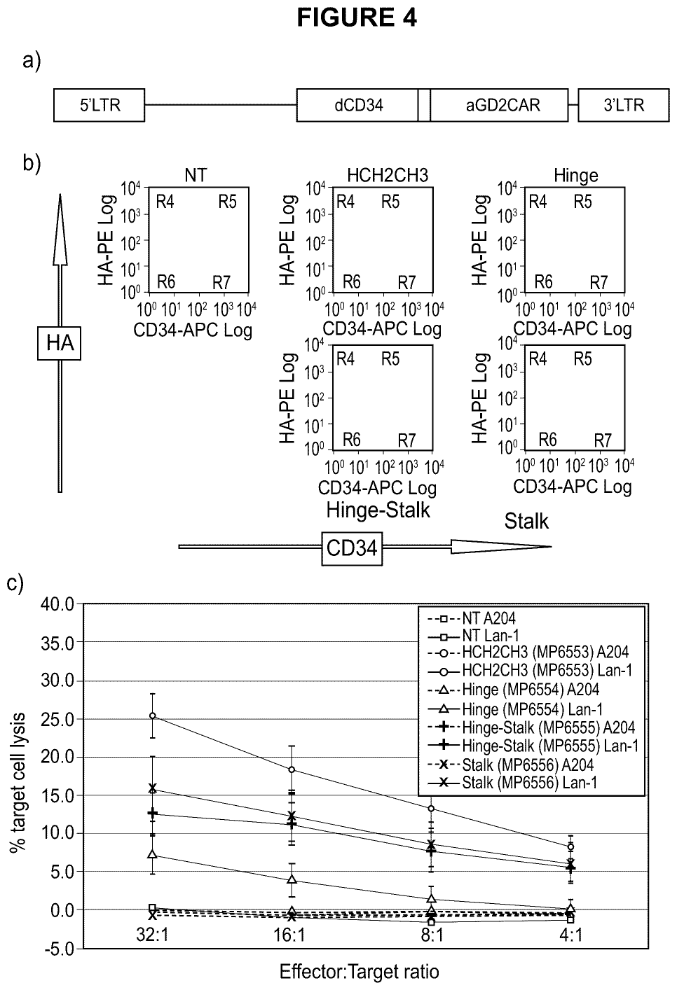

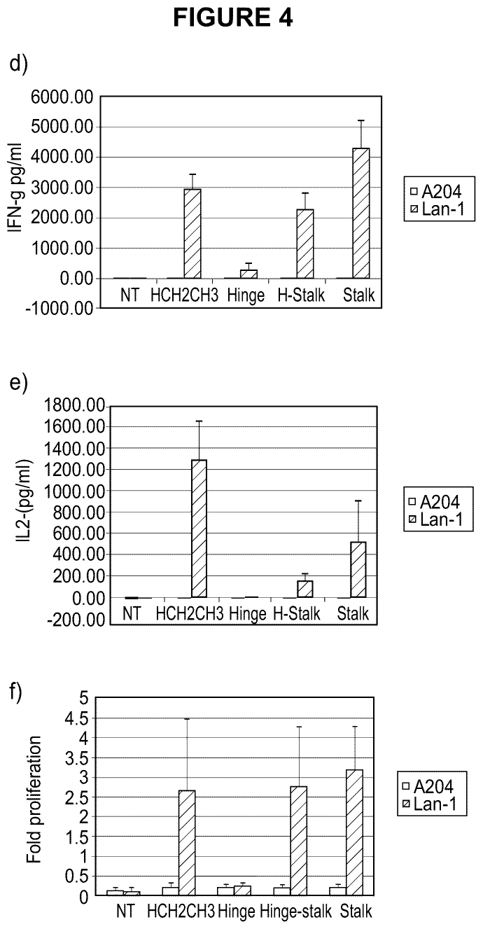

Anti-GD2 CARs with Fc spacer, Hinge, Hinge-CD8 stalk and Cd8 stalk were generated (FIGS. 2 (b), (d), (e) and (f) respectively). These CARs were co-expressed with the marker gene, truncated CD34, in an obligate 1:1 fashion with the 2A foot-and-mouth self-cleaving peptide to allow accurate comparison (FIG. 4a). Further, the huk666 scFv was tagged with an aminoterminal HA tag to allow comparison of transgene versus CAR expression.

Flow cytometric analysis of normal donor T-cells transduced with these constructs demonstrated brighter CAR expression in the following order: Fc>Hinge-stalk=stalk>Hinge (FIG. 4b).

Killing of GD2 positive targets relative to GD2 negative targets was compared using chromium release assays. This showed killing effectiveness in the following order: Fc>Hinge-stalk=stalk>Hinge (FIG. 4c).

Interferon-gamma release and IL-2 release was compared when CAR T-cells were challenged with either GD2 positive or negative targets. Interferon-gamma release was similar in CARs with Fc, hinge-stalk and stalk but less in the hinge variant. IL2 release was detected in the following order: Fc, stalk, hinge-stalk, hinge (FIGS. 4d and e).

Finally, proliferation of CAR T-cells was compared when CAR T-cells were challenged with either GD2 positive or negative targets. Proliferation was detected in the following order: Stalk, hinge-stalk, Fc, hinge (FIGS. 4d and e)

### Example 3—FcR Mutations Abrogate Non-Specific Activity

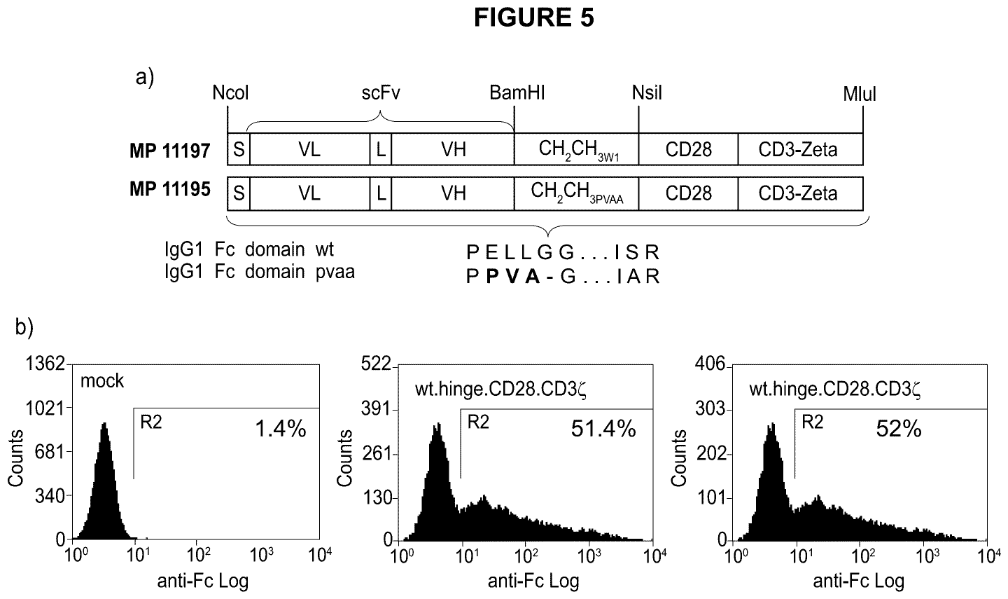

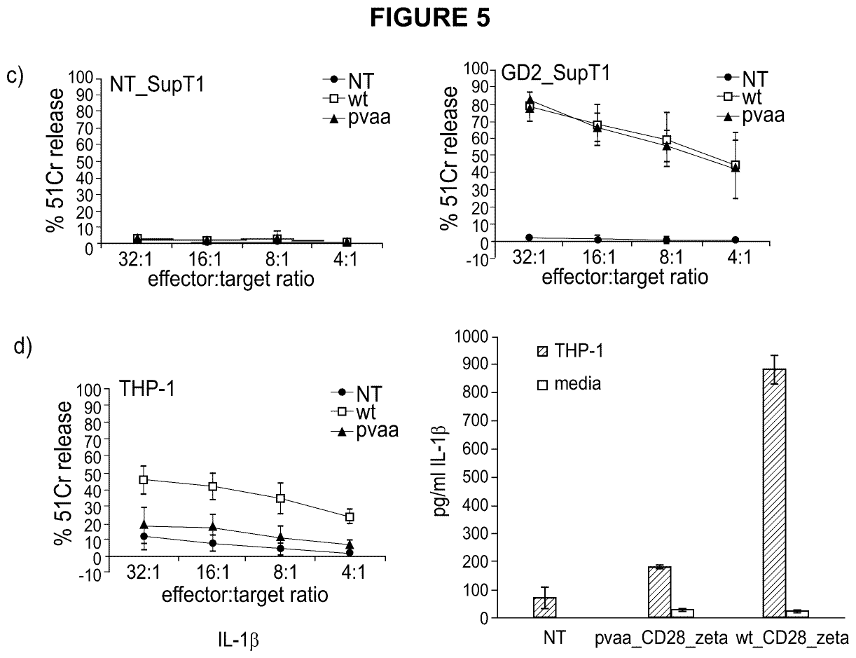

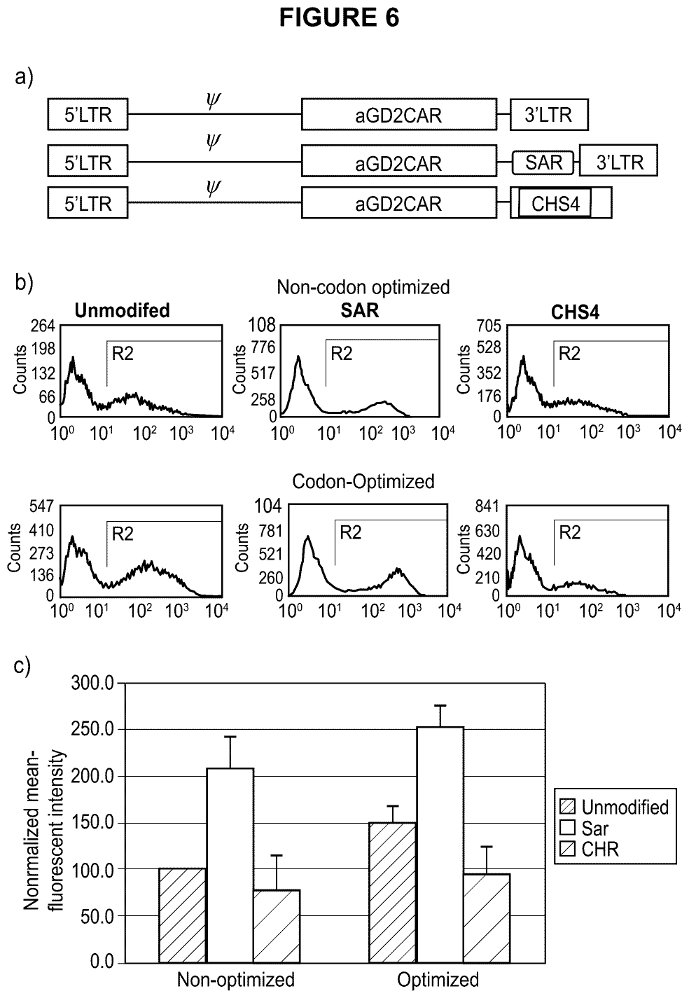

The overall data from the above Examples suggested that the Fc spacer performs best overall. However the Fc domain in vivo may lead to non-specific activation from cells which express Fc receptors. To abrogate this effect, mutations were introduced into the Fc region as shown in FIG. 5(a). These mutations had no deleterious effects on CAR expression, as shown in FIG. 5(b).

In addition, it was shown that these mutations had no effect on CAR killing function (FIG. 5(c)). Finally, it was shown that these mutations had the desired effect in terms of non-specific killing of FcR expressing targets (a monocytoid line called THP1), and IL-1Beta release by these monocytes (FIG. 5e).

### Example 4—Optimization of the Expression Cassette

With a view to optimising expression of the receptor, the following were tested: (a) inclusion of a scaffold attachment region (SAR) into the cassette; (b) inclusion of chicken beta hemoglobin chromatin insulator (CHS4) into the 3′LTR and (c) codon optimization of the open reading frame (FIG. 6a). It was shown that inclusion of a SAR improved the nature of expression as did codon-optimization while the CHS4 had little effect (FIG. 6b). Combining SAR and codon-optimization improved expression additively (FIG. 6c)

### Example 5—Comparison of Different Endodomains

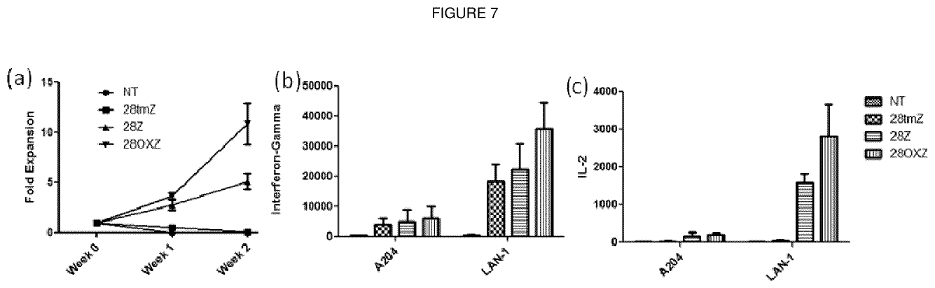

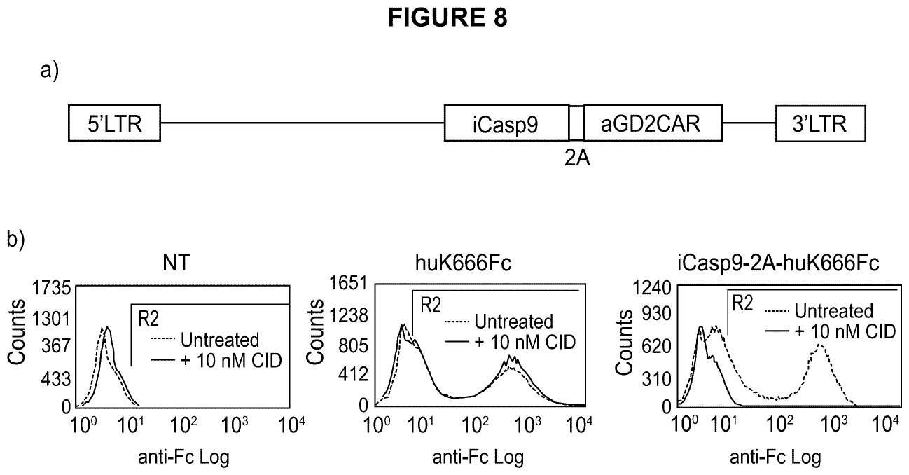

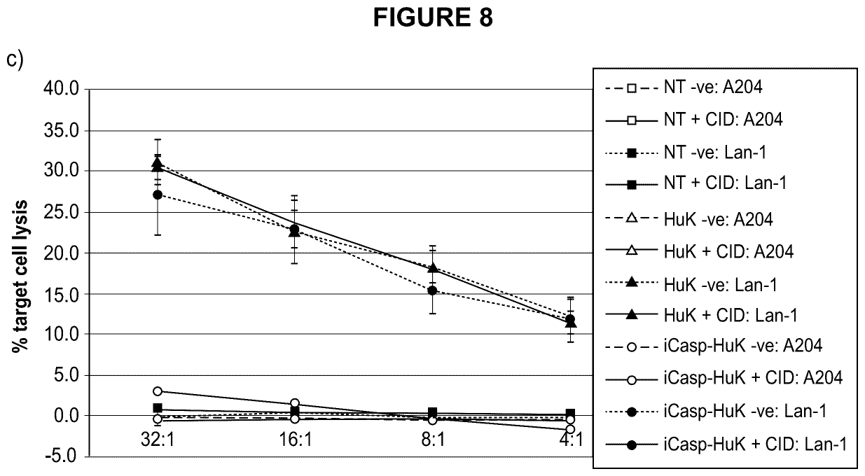

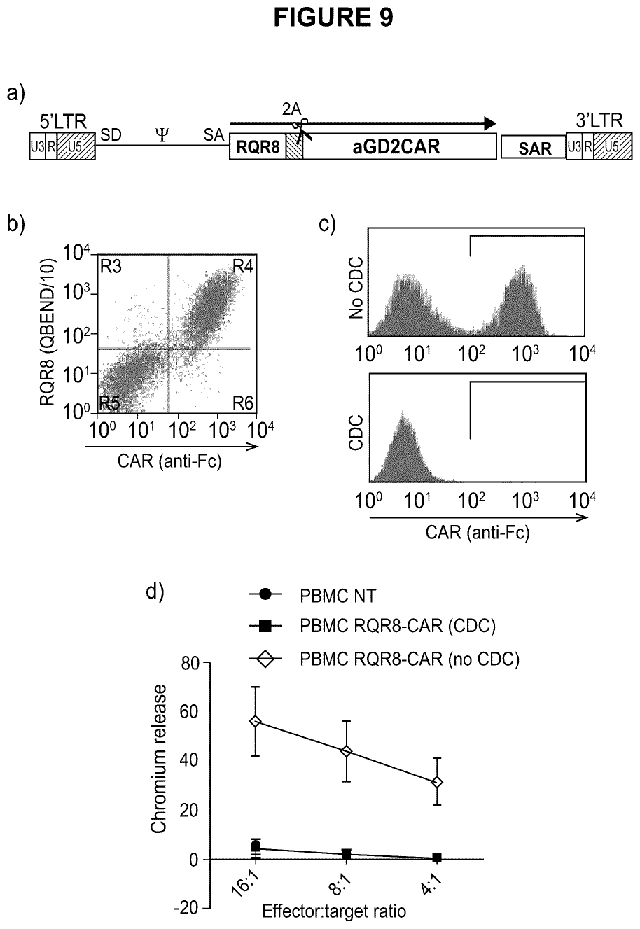

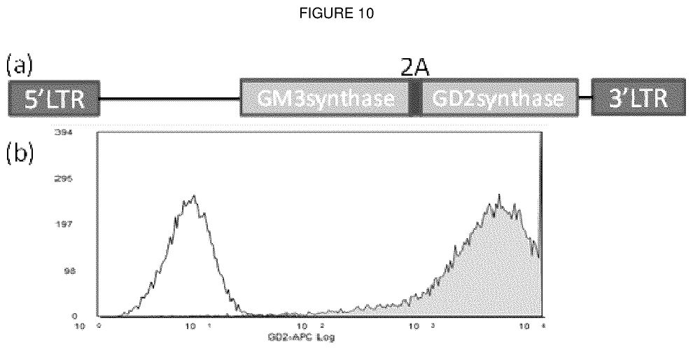

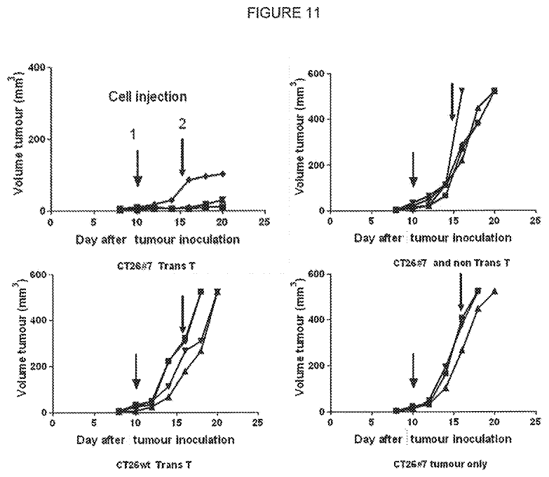

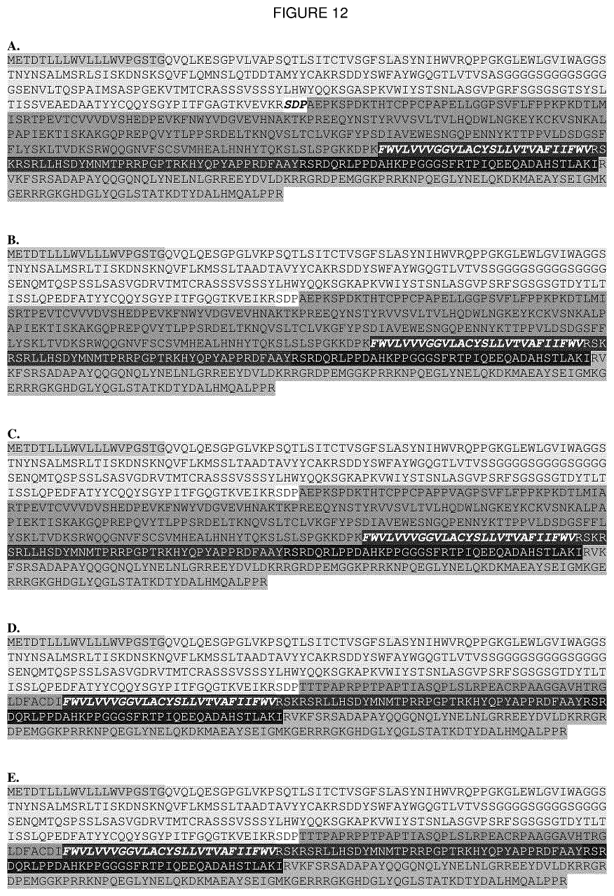

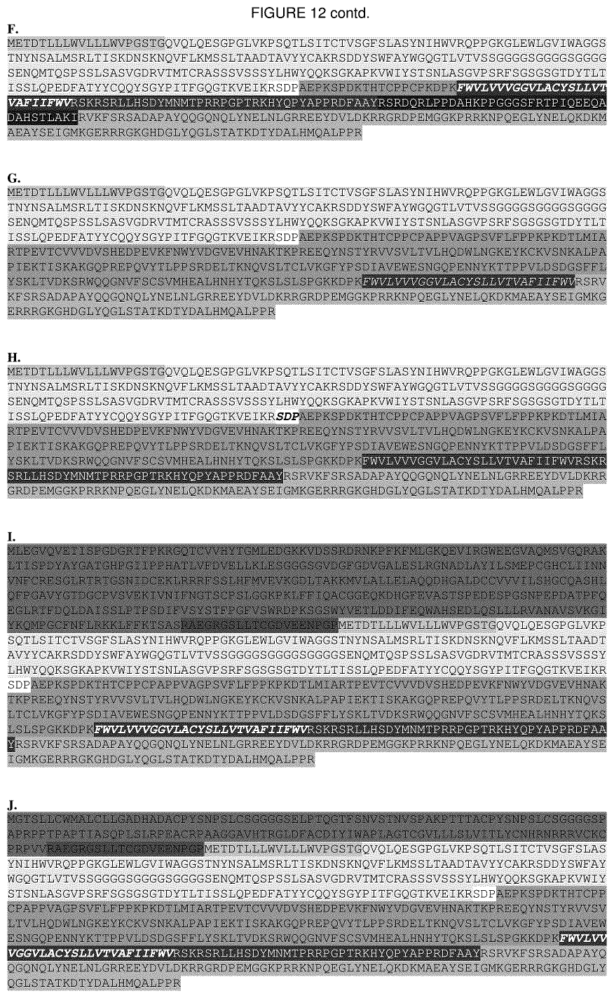

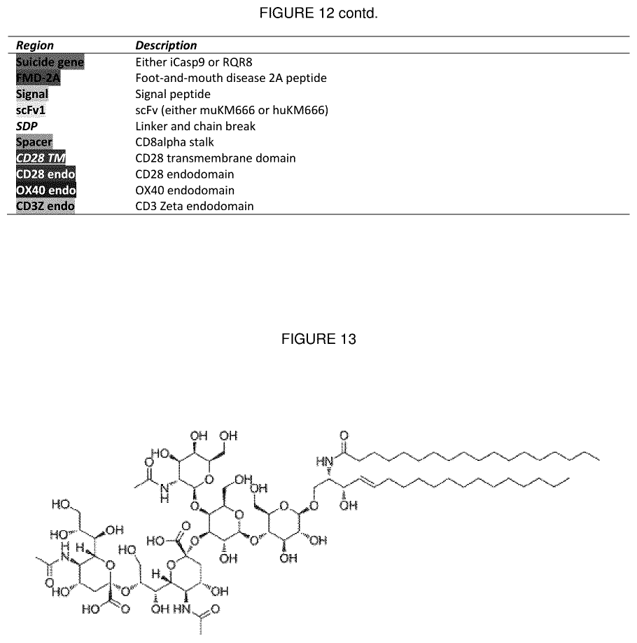

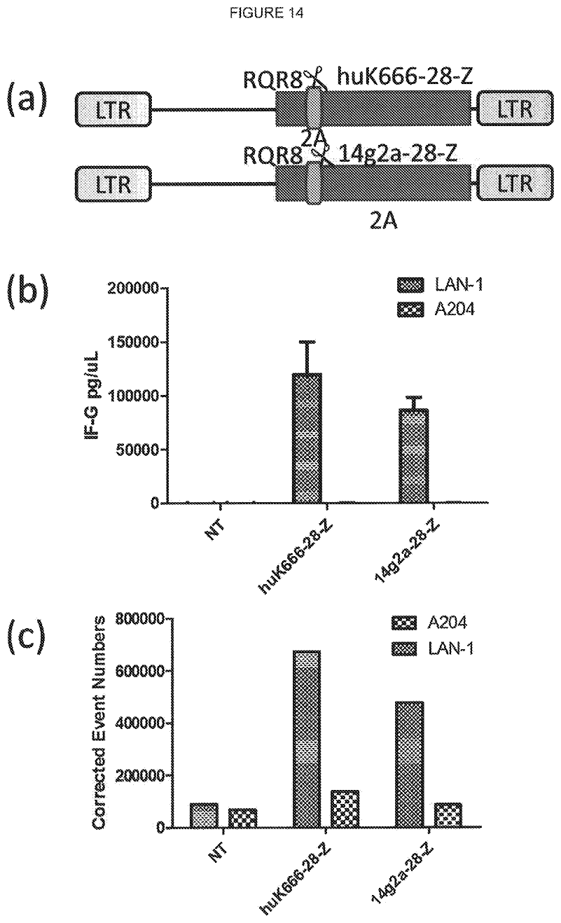

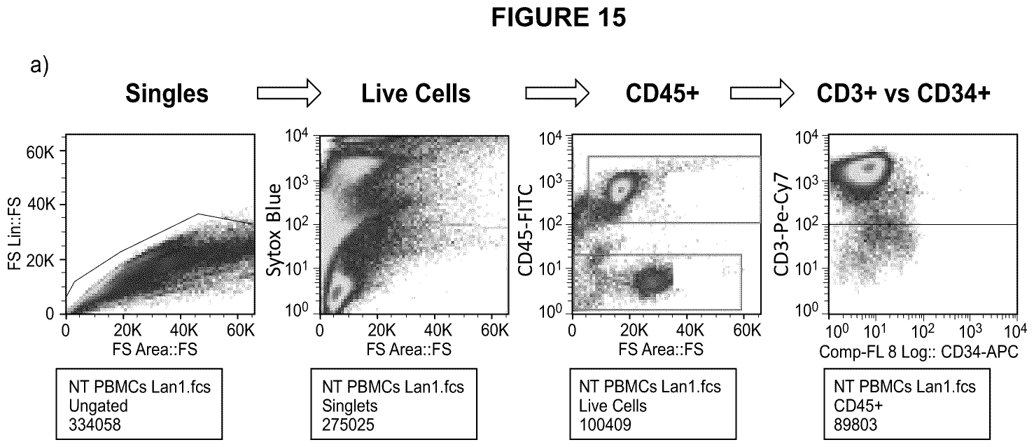

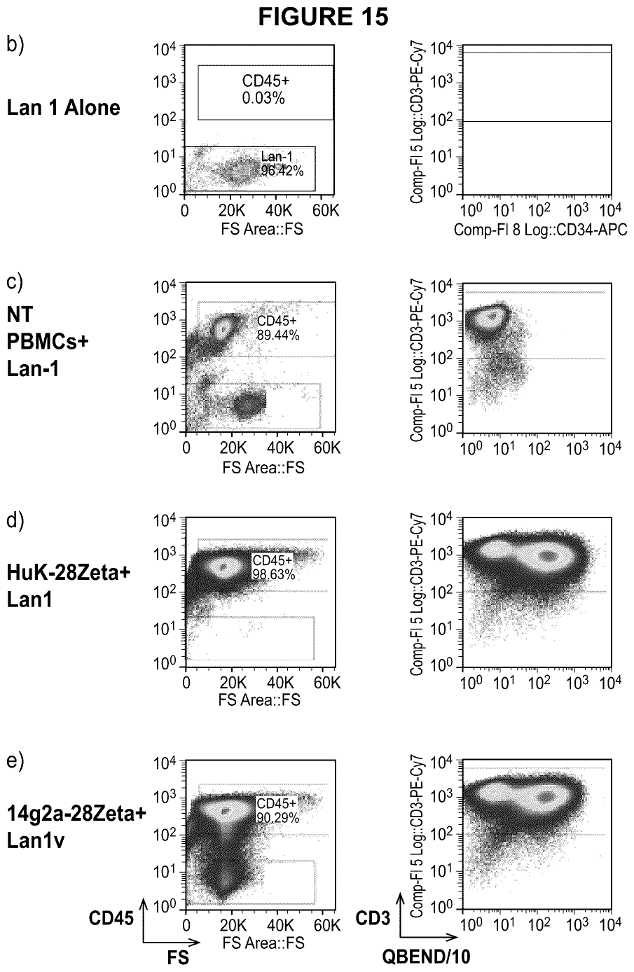

Constructs with three different endodomains were generated: CD28 trans-membrane domain with CD3-zeta endodomain (CD28tmZ); CD28 transmembrane domain with CD28 endodomain and CD3-zeta endodomain (CD28Z), and CD28 transmembrane domain, CD28 endodomain, OX30 endodomain and CD3-zeta endodomain (CD28OXZ) with a CAR in the Fc spacer format. Proliferation, IFNγ release and IL-2 release were noted to increase in order of CD28tmZ<CD28Z<CD28OXZ (FIG. 7).

### Example 6—Co-Expression with iCasp9 Suicide Gene

The iCasp9 suicide gene was co-expressed with the anti-GD2 CAR (FIG. 8a—the CAR was in the format of Fc-spacer, CD28OXZ chosen arbitrarily to demonstrate function). The CAR could be well-expressed despite co-expression with iCasp9 (FIG. 8b). Activation of iCasp9 with the small molecular dimerizer led to deletion of CAR positive T-cells (FIG. 8b). iCasp9-GD2CAR T-cells exposed to this dimerizer lost their GD2 specificity when exposed to the dimerizer (FIG. 8c).

### Example 7—Co-Expression with RQR8 Suicide Gene

The anti-GD2 CAR was co-expressed with the RQR8 sort-suicide gene. (FIG. 9a—the CAR was in the format of Fc-spacer, CD28Z chosen arbitrarily to demonstrate function). It was possible to co-express receptor and CAR (FIG. 9b). Activation of the suicide gene function of RQR8 with Rituximab and complement resulted in deletion of transduced T-cells and loss of GD2 recognition (FIG. 9 c and d).

### Example 8—Expression of GD2 Synthase and GM3 Synthase Results in GD2 Expression in any Cell Line

In order to stimulate GD2CAR T-cells in culture, to have ideal GD2− or GD2+ targets, and to be able to generate syngeneic cells for small animal models, it is desirable to be able to transgenically express GD2 on a cell line. GD2 is not a protein and needs to be synthesized by a complex set of enzymes. Here it is shown that transgenic expression expression of just two enzymes: GM3synthase and GD2synthase results in bright GD2 expression in all cell lines transduced thus far (FIG. 10).

### Example 9—In Vivo Function of Anti-GD2 CAR

CT26 cell line was engineered to express GD2 as described above (designated CT26 clone #7 or CT25 #7 for short). Either 2×10⁵ of wild type (wt) or GD2 positive CD26 cells were inoculated into the flanks of C57BL/6 mice (syngeneic with CT26). 10 days after tumour challenge, mock-transduced and anti-GD2 CAR transduced syngeneic splenocytes were prepared. Mice were divided into the following 4 cohorts: mice with GD2 expressing CT26 tumours receiving anti-GD2 CAR spleoncytes; GD2 expressing CT26 tumours receiving mock-transduced splenocytes; GD2 negative (wt) CT26 tumours with anti-GD2 CAR splenocytes; and GD2 expressing CT26 tumours receiving no splenocytes. Tumour was measured using a digital caliper in 3 dimension and volume estimated therewith. FIG. 11 shows the growth curves of the tumours. Only GD2 positive tumours in mice receiving anti-GD2 CAR T-cells had little or no growth.

### Example 10—Comparing the Function of CARs Comprising Huk666 and 14g2a-Based Antigen Binding Domains

The antigen binding domain of a CAR can affect its function. In this study, the function of the CAR of the invention with an antigen-binding domain based on huk666 with a CAR was compared with an equivalent CAR having an antigen-binding domain based on 14g2a.

The antibody 14g2a can be seen as the gold standard antibody against GD2 since it is used as a therapeutic mAb and it is the only scFv tested in a CAR study.

Second generation CARs were constructed and expressed based on huk666 or 14g2a. Their structure is shown in FIG. 14a.

Retroviruses were produced by transient transfection of 293T cells with plasmids encoding the GD2 CARs, gag/pol and the envelope protein RD114. After 3 days the supernatants were harvested and used to transduce PHA/IL2-activated PBMCs with equal titres of retrovirus on retronectin-coated plates. The CARs differed solely in their antigen binding domain. In both cases the binding domains were linked to the membrane with an IgG Fc segment and contained intracellular activatory motifs from CD28 and CD3-zeta. Six days post-transduction CAR-expression was confirmed by flow cytometry and PBMCs were cultured in a 1:1 ratio with GD2-positive Lan1 cells (a GD2 positive cell line) or GD2-negative A204 cells (a GD2 negative rhabdomyosarcoma cell line). After one day supernatants from these co-cultures were assayed for interferon-γ levels by ELISA and T cell proliferation was assessed by flow cytometry after 6 days.

The results are shown in FIGS. 14 and 15. At 24 hours, Interferon-gamma was measured from supernatant. huK666 CAR T-cells were shown to produce more IFN-γ (FIG. 14b). After one week T-cells were counted, and the huk666 CAR was show to have more proliferation (FIG. 14c).

After one week of co-culture with the Neuroblastoma cell line LAN1, cells were harvested and analyzed by flow-cytometry. CD45 expression allowed discrimination from lymphoid cells and non-lymphoid cells with CD45-cells being LAN-1 cells. Further staining with CD3/QBEND/10 allowed counting of CAR T-cells. It was found that huk666 CAR T-cells proliferate better and kill more completely than 14g2a equivalents (FIG. 15).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A chimeric antigen receptor (CAR) comprising a disialoganglioside (GD2)-binding domain which comprises
a) a heavy chain variable region (VH) having complementarity determining regions (CDRs) with the following sequences:
CDR1
(SEQ ID No. 1)
SYNIH;
CDR2
(SEQ ID No. 2)
VIWAGGSTNYNSALMS
CDR3
(SEQ ID No. 3)
RSDDYSWFAY;
and
b) a light chain variable region (VL) having CDRs with the following sequences:
CDR1
(SEQ ID No. 4)
RASSSVSSSYLH;
CDR2
(SEQ ID No. 5)
STSNLAS
CDR3
(SEQ ID No. 6)
QQYSGYPIT.

2. A CAR according to claim 1, wherein the GD2 binding domain comprises a VH domain having the sequence shown as SEQ ID No. 9, or SEQ ID NO 10; or a VL domain having the sequence shown as SEQ ID No 11, or SEQ ID No. 12 or a variant thereof having at least 90% sequence identity which retains the capacity to i) bind GD2 and ii) induce T cell signalling.

3. A CAR according to claim 1, wherein the GD2 binding domain comprises the sequence shown as SEQ ID No 7 or SEQ ID No. 8 or a variant thereof having at least 90% sequence identity which retains the capacity to i) bind GD2 and ii) induce T cell signalling.

4. A CAR according to claim 1, which comprises a transmembrane domain which comprises the sequence shown as SEQ ID No. 13 or a variant thereof having at least 80% sequence identity.

5. A CAR according to claim 1, wherein GD2-binding domain and the transmembrane domain are connected by a spacer.

6. A CAR according to claim 4, wherein the spacer comprises one of the following: a human an IgG1 Fc domain; an IgG1 hinge; an IgG1 hinge-CD8 stalk; or a CD8 stalk.

7. A CAR according to claim 6, wherein the spacer comprises an IgG1 hinge-CD8 stalk or a CD8 stalk.

8. A CAR according to claim 6, wherein the spacer comprises an IgG1 Fc domain or a variant thereof.

9. A CAR according to claim 8, wherein the spacer comprises an IgG1 Fc domain which comprises the sequence shown as SEQ ID No. 23 or SEQ ID No. 24 or a variant thereof having at least 80% sequence identity.

10. A CAR according to claim 1 which also comprises an intracellular T cell signalling domain.

11. A CAR according to claim 10 wherein the intracellular T cell signalling domain comprises one or more of the following endodomains: CD28 endodomain; OX40 and CD3-Zeta endodomain.

12. A CAR according to claim 11 wherein the intracellular T cell signalling domain comprises all of the following endodomains: CD28 endodomain; OX40 and CD3-Zeta endodomain.

13. A CAR according to claim 1, which comprises the sequence shown as any of SEQ ID No. 26 to 35 or a variant thereof which has at least 80% sequence identity but retains the capacity to i) bind GD2 and ii) induce T cell signalling.

14. A nucleic acid sequence which encodes a CAR according to claim 1.

15. A nucleic acid sequence according to claim 14, which is codon-optimised.

16. A nucleic acid sequence according to claim 14 which comprises the sequence shown as SEQ ID No 25 or a variant thereof having at least 90% sequence identity.

17. A nucleic acid according to any of claim 14 which also encodes a suicide gene.

18. A vector which comprises a nucleic acid sequence according to claim 14.

19. A T cell which expresses a CAR according to claim 1.

20. A T cell which co-expresses a CAR according to to claim 1 and a suicide gene.

21. A T cell according to claim 20, wherein the suicide gene is iCasp9 or RQR8.

22. A method for making a T cell, which comprises the step of introducing a nucleic acid according to claim 14 into a T cell.

23. A pharmaceutical composition which comprises a T cell according to claim 19, together with a pharmaceutically acceptable carrier, diluent or excipient.

24. A method for treating cancer which comprises the step of administering a T cell according to claim 19 to a subject.

25. A method according to claim 24, wherein the cancer is neuroblastoma.

26-27. (canceled)


SEQUENCE LISTING (37 sequences)

SEQ ID NO: 1 - protein, 5 aa, synthetic construct
  REGION 1..5: note = Heavy chain variable region CDR1
SYNIH

SEQ ID NO: 2 - protein, 16 aa, synthetic construct
  REGION 1..16: note = Heavy chain variable region CDR2
VIWAGGSTNYNSALMS

SEQ ID NO: 3 - protein, 10 aa, synthetic construct
  REGION 1..10: note = Heavy chain variable region CDR3
RSDDYSWFAY

SEQ ID NO: 4 - protein, 12 aa, synthetic construct
  REGION 1..12: note = Light chain variable region CDR1
RASSSVSSSYLH

SEQ ID NO: 5 - protein, 7 aa, synthetic construct
  REGION 1..7: note = Light chain variable region CDR2
STSNLAS

SEQ ID NO: 6 - protein, 9 aa, synthetic construct
  REGION 1..9: note = Light chain variable region CDR3
QQYSGYPIT

SEQ ID NO: 7 - protein, 243 aa, synthetic construct
  REGION 1..243: note = Murine KM666 sequence
QVQLKESGPVLVAPSQTLSITCTVSGFSLASYNIHWVRQPPGKGLEWLGVIWAGGSTNYNSALMSRLSISKDNSKSQVFLQMNSLQTDDTAMYYCAKRSDDYSWFAYWGQGTLVTVSASGGGGSGGGGSGGGGSENVLTQSPAIMSASPGEKVTMTCRASSSVSSSYLHWYQQKSGASPKVWIYSTSNLASGVPGRFSGSGSGTSYSLTISSVEAEDAATYYCQQYSGYPITFGAGTKVEVKR

SEQ ID NO: 8 - protein, 242 aa, synthetic construct
  REGION 1..242: note = Humanised KM666 sequence
QVQLQESGPGLVKPSQTLSITCTVSGFSLASYNIHWVRQPPGKGLEWLGVIWAGGSTNYNSALMSRLTISKDNSKNQVFLKMSSLTAADTAVYYCAKRSDDYSWFAYWGQGTLVTVSSGGGGSGGGGSGGGGSENQMTQSPSSLSASVGDRVTMTCRASSSVSSSYLHWYQQKSGKAPKVWIYSTSNLASGVPSRFSGSGSGTDYTLTISSLQPEDFATYYCQQYSGYPITFGQGTKVEIKR

SEQ ID NO: 9 - protein, 118 aa, synthetic construct
  REGION 1..118: note = Murine KM666 VH (heavy chain variable region) sequence
QVQLKESGPVLVAPSQTLSITCTVSGFSLASYNIHWVRQPPGKGLEWLGVIWAGGSTNYNSALMSRLSISKDNSKSQVFLQMNSLQTDDTAMYYCAKRSDDYSWFAYWGQGTLVTVSA

SEQ ID NO: 10 - protein, 118 aa, synthetic construct
  REGION 1..118: note = Humanised KM666 VH sequence
QVQLQESGPGLVKPSQTLSITCTVSGFSLASYNIHWVRQPPGKGLEWLGVIWAGGSTNYNSALMSRLTISKDNSKNQVFLKMSSLTAADTAVYYCAKRSDDYSWFAYWGQGTLVTVSS

SEQ ID NO: 11 - protein, 108 aa, synthetic construct
  REGION 1..108: note = Murine KM666 VL (light chain variable region) sequence
ENVLTQSPAIMSASPGEKVTMTCRASSSVSSSYLHWYQQKSGASPKVWIYSTSNLASGVPGRFSGSGSGTSYSLTISSVEAEDAATYYCQQYSGYPITFGAGTKVEVK

SEQ ID NO: 12 - protein, 108 aa, synthetic construct
  REGION 1..108: note = Humanised KM666 VL sequence
ENQMTQSPSSLSASVGDRVTMTCRASSSVSSSYLHWYQQKSGKAPKVWIYSTSNLASGVPSRFSGSGSGTDYTLTISSLQPEDFATYYCQQYSGYPITFGQGTKVEIK

SEQ ID NO: 13 - protein, 27 aa, synthetic construct
  REGION 1..27: note = Transmembrane domain
FWVLVVVGGVLACYSLLVTVAFIIFWV

SEQ ID NO: 14 - protein, 39 aa, synthetic construct
  REGION 1..39: note = CD28 endodomain
RSKRSRLLHSDYMNMTPRRPGPTRKHYQPYAPPRDFAAY

SEQ ID NO: 15 - protein, 38 aa, synthetic construct
  REGION 1..38: note = CD40 endodomain
RSRDQRLPPDAHKPPGGGSFRTPIQEEQADAHSTLAKI

SEQ ID NO: 16 - protein, 114 aa, synthetic construct
  REGION 1..114: note = CD3 zeta endodomain
RSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 153 aa, synthetic construct
  REGION 1..153: note = CD28Z
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 189 aa, synthetic construct
  REGION 1..189: note = CD28OXZ
RSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 20 aa, synthetic construct
  REGION 1..20: note = Signal peptide
METDTLLLWVLLLWVPGSTG

SEQ ID NO: 20 - protein, 234 aa, synthetic construct
  REGION 1..234: note = Hinge-CH2CH3 of human IgG1 spacer
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 21 - protein, 46 aa, synthetic construct
  REGION 1..46: note = Human CD8 stalk spacer
TTTPAPRPPTPAPTIASQPLSLRPEACRPAAGGAVHTRGLDFACDI

SEQ ID NO: 22 - protein, 20 aa, synthetic construct
  REGION 1..20: note = Human IgG1 hinge spacer
AEPKSPDKTHTCPPCPKDPK

SEQ ID NO: 23 - protein, 237 aa, synthetic construct
  REGION 1..237: note = IgG1 Hinge-Fc spacer
AEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 24 - protein, 236 aa, synthetic construct
  REGION 1..236: note = IgG1 Hinge spacer - Fc modified to remove Fc receptor recognition motifs
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 25 - DNA, 6014 bp, synthetic construct
  misc_feature 1..6014: note = Retroviral cassette
  source 1..6014: mol_type = other DNA
tgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccaacatgggcaccagcctgctgtgctggatggccctgtgcctgctgggcgccgaccacgccgatgcctgcccctacagcaaccccagcctgtgcagcggaggcggcggcagcgagctgcccacccagggcaccttctccaacgtgtccaccaacgtgagcccagccaagcccaccaccaccgcctgtccttattccaatccttccctgtgtagcggagggggaggcagcccagcccccagacctcccaccccagcccccaccatcgccagccagcctctgagcctgagacccgaggcctgccgcccagccgccggcggcgccgtgcacaccagaggcctggatttcgcctgcgatatctacatctgggccccactggccggcacctgtggcgtgctgctgctgagcctggtgatcaccctgtactgcaaccaccgcaaccgcaggcgcgtgtgcaagtgccccaggcccgtggtgagagccgagggcagaggcagcctgctgacctgcggcgacgtggaggagaacccaggccccatggagaccgacaccctgctgctgtgggtgctgctgctgtgggtgccaggcagcaccggccaggtgcagctgcaggagtctggcccaggcctggtgaagcccagccagaccctgagcatcacctgcaccgtgagcggcttcagcctggccagctacaacatccactgggtgcggcagcccccaggcaagggcctggagtggctgggcgtgatctgggctggcggcagcaccaactacaacagcgccctgatgagccggctgaccatcagcaaggacaacagcaagaaccaggtgttcctgaagatgagcagcctgacagccgccgacaccgccgtgtactactgcgccaagcggagcgacgactacagctggttcgcctactggggccagggcaccctggtgaccgtgagctctggcggaggcggctctggcggaggcggctctggcggaggcggcagcgagaaccagatgacccagagccccagcagcttgagcgccagcgtgggcgaccgggtgaccatgacctgcagagccagcagcagcgtgagcagcagctacctgcactggtaccagcagaagagcggcaaggccccaaaggtgtggatctacagcaccagcaacctggccagcggcgtgcccagccggttcagcggcagcggcagcggcaccgactacaccctgaccatcagcagcctgcagcccgaggacttcgccacctactactgccagcagtacagcggctaccccatcaccttcggccagggcaccaaggtggagatcaagcggtcggatcccgccgagcccaaatctcctgacaaaactcacacatgcccaccgtgcccagcacctcccgtggccggcccgtcagtcttcctcttccccccaaaacccaaggacaccctcatgatcgcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggccctgcacaatcactatacccagaaatctctgagtctgagcccaggcaagaaggaccccaagttctgggtcctggtggtggtgggaggcgtgctggcctgttactctctcctggtgaccgtggccttcatcatcttctgggtgcgctccaagaggagcaggctcctgcacagtgactacatgaacatgactccccgccgccccgggcccacccgcaagcattaccagccctatgccccaccacgcgacttcgcagcctatcgctcccgggtgaagttctctcgctctgccgatgccccagcctatcagcagggccagaatcagctgtacaatgaactgaacctgggcaggcgggaggagtacgacgtgctggataagcggagaggcagagaccccgagatgggcggcaaaccacggcgcaaaaatccccaggagggactctataacgagctgcagaaggacaaaatggccgaggcctattccgagatcggcatgaagggagagagaagacgcggaaagggccacgacggcctgtatcagggattgtccaccgctacaaaagatacatatgatgccctgcacatgcaggccctgccacccagatgacgcgtatcgatactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttcggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcac

SEQ ID NO: 26 - protein, 719 aa, synthetic construct
  REGION 1..719: note = anti-GD2 CAR, muKM666-HCH2CH3-CD28OXZ
METDTLLLWVLLLWVPGSTGQVQLKESGPVLVAPSQTLSITCTVSGFSLASYNIHWVRQPPGKGLEWLGVIWAGGSTNYNSALMSRLSISKDNSKSQVFLQMNSLQTDDTAMYYCAKRSDDYSWFAYWGQGTLVTVSASGGGGSGGGGSGGGGSENVLTQSPAIMSASPGEKVTMTCRASSSVSSSYLHWYQQKSGASPKVWIYSTSNLASGVPGRFSGSGSGTSYSLTISSVEAEDAATYYCQQYSGYPITFGAGTKVEVKRSDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 718 aa, synthetic construct
  REGION 1..718: note = anti-GD2 CAR, huKM666-HCH2CH3-CD28OXZ
METDTLLLWVLLLWVPGSTGQVQLQESGPGLVKPSQTLSITCTVSGFSLASYNIHWVRQPPGKGLEWLGVIWAGGSTNYNSALMSRLTISKDNSKNQVFLKMSSLTAADTAVYYCAKRSDDYSWFAYWGQGTLVTVSSGGGGSGGGGSGGGGSENQMTQSPSSLSASVGDRVTMTCRASSSVSSSYLHWYQQKSGKAPKVWIYSTSNLASGVPSRFSGSGSGTDYTLTISSLQPEDFATYYCQQYSGYPITFGQGTKVEIKRSDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 717 aa, synthetic construct
  REGION 1..717: note = anti-GD2 CAR, huKM666-HCH2CH3pvaa-CD28OXZ
METDTLLLWVLLLWVPGSTGQVQLQESGPGLVKPSQTLSITCTVSGFSLASYNIHWVRQPPGKGLEWLGVIWAGGSTNYNSALMSRLTISKDNSKNQVFLKMSSLTAADTAVYYCAKRSDDYSWFAYWGQGTLVTVSSGGGGSGGGGSGGGGSENQMTQSPSSLSASVGDRVTMTCRASSSVSSSYLHWYQQKSGKAPKVWIYSTSNLASGVPSRFSGSGSGTDYTLTISSLQPEDFATYYCQQYSGYPITFGQGTKVEIKRSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 527 aa, synthetic construct
  REGION 1..527: note = anti-GD2 CAR, huKM666-HSTK-CD28OXZ
METDTLLLWVLLLWVPGSTGQVQLQESGPGLVKPSQTLSITCTVSGFSLASYNIHWVRQPPGKGLEWLGVIWAGGSTNYNSALMSRLTISKDNSKNQVFLKMSSLTAADTAVYYCAKRSDDYSWFAYWGQGTLVTVSSGGGGSGGGGSGGGGSENQMTQSPSSLSASVGDRVTMTCRASSSVSSSYLHWYQQKSGKAPKVWIYSTSNLASGVPSRFSGSGSGTDYTLTISSLQPEDFATYYCQQYSGYPITFGQGTKVEIKRSDPTTTPAPRPPTPAPTIASQPLSLRPEACRPAAGGAVHTRGLDFACDIFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 527 aa, synthetic construct; identical to SEQ ID NO: 29
  REGION 1..527: note = anti-GD2 CAR, huKM666-STK-CD28XOXZ

SEQ ID NO: 31 - protein, 501 aa, synthetic construct
  REGION 1..501: note = anti-GD2 CAR, huKM666-HNG-CD28OXZ
METDTLLLWVLLLWVPGSTGQVQLQESGPGLVKPSQTLSITCTVSGFSLASYNIHWVRQPPGKGLEWLGVIWAGGSTNYNSALMSRLTISKDNSKNQVFLKMSSLTAADTAVYYCAKRSDDYSWFAYWGQGTLVTVSSGGGGSGGGGSGGGGSENQMTQSPSSLSASVGDRVTMTCRASSSVSSSYLHWYQQKSGKAPKVWIYSTSNLASGVPSRFSGSGSGTDYTLTISSLQPEDFATYYCQQYSGYPITFGQGTKVEIKRSDPAEPKSPDKTHTCPPCPKDPKFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 642 aa, synthetic construct
  REGION 1..642: note = anti-GD2 CAR, huKM666-HCH2CH3pvaa-CD28tmZ
METDTLLLWVLLLWVPGSTGQVQLQESGPGLVKPSQTLSITCTVSGFSLASYNIHWVRQPPGKGLEWLGVIWAGGSTNYNSALMSRLTISKDNSKNQVFLKMSSLTAADTAVYYCAKRSDDYSWFAYWGQGTLVTVSSGGGGSGGGGSGGGGSENQMTQSPSSLSASVGDRVTMTCRASSSVSSSYLHWYQQKSGKAPKVWIYSTSNLASGVPSRFSGSGSGTDYTLTISSLQPEDFATYYCQQYSGYPITFGQGTKVEIKRSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 681 aa, synthetic construct
  REGION 1..681: note = anti-GD2 CAR, huMK666-HCH2CH3pvaa-CD28Z
METDTLLLWVLLLWVPGSTGQVQLQESGPGLVKPSQTLSITCTVSGFSLASYNIHWVRQPPGKGLEWLGVIWAGGSTNYNSALMSRLTISKDNSKNQVFLKMSSLTAADTAVYYCAKRSDDYSWFAYWGQGTLVTVSSGGGGSGGGGSGGGGSENQMTQSPSSLSASVGDRVTMTCRASSSVSSSYLHWYQQKSGKAPKVWIYSTSNLASGVPSRFSGSGSGTDYTLTISSLQPEDFATYYCQQYSGYPITFGQGTKVEIKRSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 1103 aa, synthetic construct
  REGION 1..1103: note = anti-GD2 CAR co-expressed with iCasp9 suicide gene
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRAEGRGSLLTCGDVEENPGPMETDTLLLWVLLLWVPGSTGQVQLQESGPGLVKPSQTLSITCTVSGFSLASYNIHWVRQPPGKGLEWLGVIWAGGSTNYNSALMSRLTISKDNSKNQVFLKMSSLTAADTAVYYCAKRSDDYSWFAYWGQGTLVTVSSGGGGSGGGGSGGGGSENQMTQSPSSLSASVGDRVTMTCRASSSVSSSYLHWYQQKSGKAPKVWIYSTSNLASGVPSRFSGSGSGTDYTLTISSLQPEDFATYYCQQYSGYPITFGQGTKVEIKRSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 858 aa, synthetic construct
  REGION 1..858: note = anti-GD2 CAR co-expressed with RQR8 suicide gene
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVRAEGRGSLLTCGDVEENPGPMETDTLLLWVLLLWVPGSTGQVQLQESGPGLVKPSQTLSITCTVSGFSLASYNIHWVRQPPGKGLEWLGVIWAGGSTNYNSALMSRLTISKDNSKNQVFLKMSSLTAADTAVYYCAKRSDDYSWFAYWGQGTLVTVSSGGGGSGGGGSGGGGSENQMTQSPSSLSASVGDRVTMTCRASSSVSSSYLHWYQQKSGKAPKVWIYSTSNLASGVPSRFSGSGSGTDYTLTISSLQPEDFATYYCQQYSGYPITFGQGTKVEIKRSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 402 aa, synthetic construct
  REGION 1..402: note = Inducible Caspase 9 (iCasp9) sequence
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 37 - protein, 157 aa, synthetic construct
  REGION 1..157: note = Novel marker/suicide gene RQR8 sequence
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV